(19) United States
(12) Patent Application Publication
(10) Pub. No.: US 2017/0319815 A1
(43) Pub. Date: Nov. 9, 2017

(54) METHOD AND APPARATUSES FOR MODULATING SLEEP BY CHEMICAL ACTIVATION OF TEMPERATURE RECEPTORS

(71) Applicant: EBB THERAPEUTICS, INC., Pittsburgh, PA (US)

(72) Inventors: Eric A. NOFZINGER, Allison Park, PA (US); Jeffrey J. SCHIRM, Monroeville, PA (US); Damian F. RIPPOLE, Coraopolis, PA (US); Craig B. REYNOLDS, Kennesaw, GA (US); Robert E. TUCKER, Sanibel, FL (US)

(21) Appl. No.: 15/521,375

(22) PCT Filed: Jan. 27, 2016
(86) PCT No.: PCT/US16/15174

§ 371 Date: Apr. 24, 2017

Related U.S. Application Data
(60) Provisional application No. 62/108,461, filed on Jan. 27, 2015.

(51) Int. Cl. A61M 21/02; A61M 37/00; A61K 9/00; A61M 21/00

(52) U.S. Cl. CPC A61M 21/02; A61K 9/0014; A61M 37/00; A61K 9/0009; A61M 2210/06; A61M 2209/088; A61M 2205/52; A61M 2205/502; A61M 2205/364; A61M 2205/3569; A61M 2205/3553; A61M 2205/3303; A61M 2037/0007; A61M 2021/0077; A61M 2021/0066; A61M 2021/0055; A61M 2021/0022; A61M 2210/083; A61M 2210/086; A61M 2230/18; A61M 2230/10

(57) ABSTRACT

Apparatuses (including devices, kits, and systems) and methods to non-invasively and chemically (rather than thermally) activate thermoreceptors to modulate sleep. For example, described herein are apparatuses including topical compositions that stimulate thermoreceptors on the subject's skin (e.g., forehead, hands, and/or feet) for a period of time to induce a sensation of temperature (heat or cold) without significantly altering the person's actual skin temperature to improve sleep quality, including reducing sleep-onset latency, enhancing depth of sleep, and/or extending the amount of time a subject sleeps. The subject may be suffering from insomnia or some other sleep disorder.

### CROSS REFERENCE TO RELATED APPLICATIONS

This application claims priority to U.S. Provisional Patent Application No. 62/108,461, filed on Jan. 27, 2015, and titled “METHOD AND APPARATUSES FOR MODULATING SLEEP BY CHEMICAL ACTIVATION OF TEMPERATURE RECEPTORS”.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The apparatus and methods described herein may be used to improve sleep, including reducing sleep onset, improving sleep maintenance, increasing sleep duration, reduce awakenings, and increasing deep sleep relative to light sleep in a subject, including a subject suffering from a disorder that affects sleep such as insomnia. Thus, the apparatuses and methods described herein may be used to treat sleeping disorders such as insomnia.

### BACKGROUND

Sufficient and high-quality sleep is important for an individual's physical, mental, and emotional health. Despite various drugs and devices on the market for enhancing sleep and treating sleep disorders, disruption and irregularities, including insomnia, resulting in poor sleep is a widespread and pervasive problem. For example, previously described devices and techniques for the treatment of sleep disorders have included the use of cooling therapies, including applying cooling therapy to a patient's forehead, to enhance sleep. This has been described, for example, in U.S. Pat. No. 8,425,583, U.S. Pat. No. 9,089,400, U.S. Pat. No. 9,211,212 and U.S. Pat. No. 8,236,038, and in pending application Ser. Nos. 14/749,590, 14/938,705, 12/288,417, 14/435,515 and 14/758,438, each of which are herein incorporated by reference in their entirety. Thus, there is evidence for enhancing sleep by cooling a subject's skin (e.g., forehead), perhaps by taking advantage of a mechanism involving cooling an underlying brain region. There is also recent work by these inventors suggesting that warming may also result in sleep enhancement, particularly warming relative to ambient temperature. The mechanism of action that temperature has on sleep has not been conclusively identified.

In general, studies have suggested that the control of sleep and thermoregulation (regulation of body temperature) are integrated at the level of the hypothalamus in the brain. Human studies have shown that manipulation of environmental temperature by various means can have an impact on sleep, however it is not well understood how selective regions of the body can influence hypothalamic sleep and thermoregulatory centers. Clinical insomnia and sleeplessness in general are characterized by transient or chronic difficulty initiating and maintaining sleep. It is unclear if alterations in thermoregulation play a significant role in the pathophysiology or treatment of insomnia or sleeplessness. Physiological and neuroanatomical studies show that the forehead is a region of the body that has unique properties and suggesting that it may play a prominent role in impacting the hypothalamic control of thermoregulation and, by extension, may influence hypothalamic thermoregulatory control of sleep.

Insomnia is often described as the inability to fall asleep easily, to stay asleep, or to experience quality sleep in an individual with adequate sleep opportunity. In the U.S., population-based estimates of either chronic (long-term) or transient (acute or short-term) insomnia range from 10% to 40% of the population, or 30 to 120 million adults. Similar prevalence estimates have been reported in Europe and Asia. Across studies, there are two age peaks for insomnia: 45-64 years of age and 85 years and older. Women are 1.3 to 2 times more likely to report trouble sleeping than men, as are those who are divorced or widowed, or have less education. In the U.S., the economic burden of insomnia approaches $100 billion in direct health care costs, functional impairment, increased risk of mental health problems, lost productivity, worker absenteeism, and excess health care utilization. Insomnia is recognized as a public health problem, contributing to more than twice the number of medical errors attributed to health care workers without insomnia episodes. Currently available treatments for insomnia are not satisfactory for a variety of reasons. Prescription drugs (e.g., sedative-hypnotics) that are given for insomnia treatment are associated with significant adverse events such as the potential for addiction/dependence, memory loss, confusional arousals, sleep walking, and problems with coordination that can lead to falls and hip fractures. The majority of insomnia patients prefer a non-pharmaceutical approach to treat their insomnia. Cognitive behavior therapy, while sometimes effective, is an expensive and labor intensive treatment that is not widely available and is not always covered by health insurance. Over-the-counter approaches to the treatment of insomnia include a variety of medications and devices that have not solved sleep problems, and inadequate clinical studies that fail to demonstrate significant effects in insomnia patients, as well as having potentially dangerous side effects. A large need exists, therefore, for a safe, effective, non-invasive treatment for treating sleep disorders and enhancing sleep.

Normal sleep cycles through different stages. The induction, maintenance and timing of wake, non-rapid eye movement sleep (NREM) or slow wave sleep (SWS), and rapid eye movement (REM) sleep stages are the result of complex interactions among multiple structures and mechanisms which are widely distributed throughout the brain. Reciprocal interactions between sleep and wake promoting systems ensure that the behavioral state of sleep-wakefulness is altered as required. Prominent among these sleep-promoting structures are the pontine tegmentum and adjacent neuronal groups in the brain that are involved in the generation of REM sleep features. On the other hand, NREM sleep is promoted by several areas in the brain, including the medial preoptic area (mPOA), the lateral preoptic area (lPOA), the ventrolateral preoptic area (vlPOA), the median preoptic nucleus (mnPO), and the medial septum, which are referred to as basal forebrain (BF) areas (Sherin et al., 1996; John and Kumar, 1998; Gong et al., 2000; Lu et al., 2000; Srividya et al., 2004, 2006). External and internal factors influence the swing of sleep-wakefulness toward either sleep or awake state. The basal forebrain plays a role in integrating thermoregulation and sleep regulation.

Body temperature regulation (thermoregulation) is a fundamental homeostatic function that is regulated by the central nervous system. The preoptic area (or POA) of the hypothalamus is considered the most important thermoregulatory site in the brain on the basis of thermoregulatory studies, such as responses elicited by local warming and cooling, analysis of lesions, results from stimulation and single neuronal recording, and other techniques. Thermoregulation in the preoptic area is controlled by thermosensitive neurons. The thermosensitive neurons in the POA receive and integrate cutaneous (skin) and deep body thermal information. These neurons are tonically active at thermoneutral temperature, and control the thermoregulatory efferent pathway.

The concept of the POA as a sleep-promoting area and the posterior hypothalamus as a wake promoting area is supported by several lines of animal experiments employing stimulation, lesion studies, single unit recording, neural transplantation, functional magnetic resonance imaging (fMRI), and c-fos studies. The neural mechanism involved in the regulation of sleep and temperature and their interrelationship has been explored in various studies.

A thermoreceptor may be a peripheral thermoreceptor (e.g., a receptor on the skin or mucous membranes of a subject that monitors external temperature) or a central thermoreceptor (e.g., an internal receptor that monitors internal body temperature). The preoptic area of the hypothalamus contains temperature sensitive neurons (warm sensitive neurons (WSN) and cold sensitive neurons (CSN)). These neurons were identified in the preoptic area on the basis of in vivo and in vitro studies. Warm sensitive neurons are directly sensitive to (and fire in response to) locally warm temperatures and cold sensitive neurons are directly sensitive to (and fire in response to) local cool temperatures.

Several studies indicate that both ambient and body temperatures influence sleep architecture. The thermoregulatory pathway which initiates a heat or cold defense response in the body is conveyed by skin thermoreceptors, en route dorsal horn, and parabrachial nuclei, to the POA. Much attention has been given to the physiological role of the POA because of its ability to control both thermoregulation and sleep. Many of the observations cited earlier support the hypothesis that sleep is modulated by thermosensitive neurons of the POA. Although this relationship has drawn considerable interest, it is still not known whether there is a “cause and effect” relationship or whether these changes are merely coincidental. Notably, studies of skin temperature in insomnia patients have focused on distal skin temperatures in the feet and hands. Whether there are other more temperature sensitive regions of the body that can transmit temperature sensitive information to the POAH is not known.

Among body regions, the forehead has unique physiological and neuroanatomical properties that suggest it may play a prominent role in influencing the thermoregulatory hypothalamic modulation of sleep. The distribution of warm and cold spots has been shown to be highest over the face and forehead of all body parts Thermal sensation has been shown to be highest in the forehead of all body parts. Further, the forehead comprising glabrous (non-hairy) skin has been shown to play a prominent role in the body response to thermoregulation given that the heat transfer function and efficacy of glabrous skin is unique within the entire body based on the capacity for a very high rate of blood perfusion and the novel capability for dynamic regulation of blood flow. Despite extensive research, it is still not well understood how sleep is controlled, and sleep disorders including insomnia, remain a significant problem for a large number of people.

Described herein are methods and apparatuses capable of stimulating a response in a subject's body to improve sleep in the subject. These methods, systems and devices may stimulate temperature sensitive receptors (e.g., cold sensitive or warm sensitive neurons) in a subject's skin to enhance the subject's sleep.

### SUMMARY OF THE DISCLOSURE

Described herein are apparatuses and methods, including methods of using the apparatuses, to enhance sleep. Enhancing sleep may include one or more of: reducing sleep onset latency, extending sleep duration, reducing awakenings, and/or increasing the duration of deeper sleep stages relative to stage 1 sleep in a subject (e.g., increasing the ratio of deeper sleep stage duration relative to stage 1 sleep duration). These apparatuses and methods may be applied to subjects (e.g., persons, patients, etc.) in need thereof; for example, the methods described herein may be used to treat a subject suffering from a sleeping disorder such as insomnia. In general, these apparatuses and methods apply (and may maintain) stimulation of temperature-sensitive receptors in a subject's body (skin) and through the receptor provide signals that enhances the subject's sleep. Also described herein are modalities that may be applied to the skin alternatively or additionally to the skin, including vibration (e.g. pulsatile mechanical force).

A temperature-sensitive receptor may be referred to herein as a thermoreceptor or thermally-responsive receptor, and may be a sensory organ or cell (e.g., a nerve cell) that is able to respond to temperature and transmit a signal about the temperature (generally to a sensory nerve). These thermoreceptors may be warm sensitive receptor(s) or cold sensitive receptor(s). While not being limited to a particular mechanism, it is generally thought that the apparatuses and methods described herein act on thermoreceptors in the skin to enhance sleep (e.g., reduce sleep onset, improve sleep maintenance, increase sleep duration, or increasing the ratio of deep sleep relative to light sleep in the subject) without requiring or directly heating/cooling the skin. Thus it is thought that they stimulate a thermally responsive receptor to have a biological response in a subject even in the absence of a specific thermal (heat or cold) stimulation. The methods and apparatus generally act by providing an applicator (e.g., containing a chemical that is a bioactive agent that activates cold-sensitive, warm-sensitive, or warm-sensitive and cold-sensitive receptors on the subject's skin) to the subject's skin that may stimulate or inhibit peripheral warm or cold sensitive neurons (thermoreceptors) in the skin. Evidence suggests that sleep and thermoregulation are integrated at the level of the hypothalamus. An intervention that targets the relationship between sleep and thermoregulation may be useful for treatment of insomnia. In some variations, a stimulating chemical (bioactive agent) may act as an agonist or antagonist to a warm sensitive receptor or a cold sensitive receptor and thus may stimulate or inhibit warm sensitive receptor(s) or cold sensitive receptor(s). Any of the bioactive agents described herein may also be referred to as signaling agents or stimulation agents as they typically stimulate (or in some cases inhibit, which is may be considered negative stimulation) thermoreceptors. A stimulating chemical may do so without causing or requiring a temperature change. For example, a stimulating chemical may be a capsaicin, a capsaicinoid, menthol, a menthol analogue, a transient receptor potential A agonist or antagonist, a TRPV1-4 (transient receptor potential vanilloid-1-4) antagonist, a TRPM 2, 4, 5, or 8 (melastatin transient receptor potential 2, 4, 5, or 8) agonist, or menthol, a vanilloid, etc.

Also described are apparatus and methods for controlling or modulating an extent or degree of stimulation (inhibit or stimulate) of a specific thermoreceptor response. A time period for stimulation (providing inhibition or activation of thermoreceptors) may be a fixed time period or a variable time period. Generally, the time period is more than 15 minutes, more than 30 min, more than 1 hour, more than 2 hours, more than 3 hours, more than 4 hours, more than 5 hours, more than 6 hours, more than 7 hours, or more than 8 hours. For example, the time period may be 30 min, 1 hour, 2 hours, 3 hours, 4 hours, 5 hours, 6 hours, 7 hours, or 8 hours or any time in between these times. The time period may be sufficiently long to cover a subject's entire sleep period or may only cover a portion of the subject's sleep period. In some variations, the apparatus and/or method may be configured to apply multiple periods of stimulation. For example, the apparatus may provide a treatment regime that includes initially applying a stimulation at a first level for a first time period (e.g., 1 hour), then increasing the stimulation to a second level for a second time period (e.g., 2 hours or more). Additional or other stimulation levels or time intervals may be applied. In some variations, the apparatus and/or method may be configured to apply different types of stimulation, such as with different types of chemicals. For example, the apparatus may provide a treatment regime that includes initially applying a first stimulation (bioactive agent) at a first level for a first time period (e.g., 5 minutes, 10 minutes, 15 minutes, 20 minutes, 25 minutes, 30 minutes, 35 minutes, 40 minutes, 45 minutes, 50 minutes, 55 minutes, 1 hour, etc.), then applying a second (e.g., a different) bioactive agent at a second level for a second time period, which may overlap with or be separate from the first time period. Additional (repeated and/or alternating) bioactive agents may be applied. As discussed below, these different chemical stimulation regimes may modulate the subject's sleep pattern and enhance sleep, including reducing sleep onset latency (the length of time to transition from full wakefulness to sleep) and reducing type 1 sleep (“light” sleep) relative to later (“deep”) sleep stages.

For example, described herein are methods of enhancing sleep in a subject having normal body temperature (e.g., no fever) including: positioning on a subject's skin an applicator having a bioactive agent configured to provide a stimulation to a cold sensitive or warm sensitive receptor; delivering the bioactive agent onto or into the subject's skin; causing a sensation of temperature with the bioactive agent without substantially changing the subject's normal body temperature with the bioactive agent; and thereby enhancing the subject's sleep.

As used herein, enhancing sleep may include an objective sleep indicator and/or a subjective sleep indicator (or both), such as one or more of: increasing the length/duration of sleep (e.g., staying asleep longer, etc.), reducing sleep latency/sleep onset, increasing the depth of sleep (e.g., increasing the time in a deep sleep state(s), such as stage 3 sleep, and/or achieving deeper stage sleep (such as increasing the duration of deeper sleep stages relative to stage 1 sleep), improving sleep quality, and improving the subjective experience of sleep. The subjective experience of sleep may include self-reported quality of a sleep indicator(s); for example, improving the subjective experience of sleep may include improving individual self-reported sleep. Thus, enhancing sleep is broadly intended to include both objective (e.g., EEG measurable data) as well as subjective (e.g., self-reported) indicators.

In general, the apparatus and methods described herein may be configured to limit the region of the body to which stimulation is applied by an applicator. This may be achieved by having an applicator configured to apply stimulation to skin regions containing responsive thermoreceptors, such as the forehead, feet or hands but does not provide a substantial amount of stimulation to other regions (such as non-responsive regions) of the body. In some examples, for the forehead, this may be achieved by configuring an applicator so that it applies the “chemical” thermoreceptor (bioactive agent) stimulation to the forehead but does not provide a substantial amount of stimulation to other, non-forehead regions of the face. In general, an applicator may avoid applying stimulation to the eye orbit region (e.g., the region beneath the eyebrows, including the periorbital and cheek regions of the face. In some examples, an applicator may be configured so that, when applied to or worn by the subject, it does not deliver a substantial amount of stimulation to the non-facial portions of the head (such as the top and back of the head). In some examples, for the foot or hand, this may be achieved by configuring an applicator so that it applies a stimulation to part of a foot or hand (such as part or all of a toe or finger, the palm, the back of the hand, etc.) but does not provide a substantial amount of stimulation to other regions of the body. Limiting the region of the face, head, foot, hand and/or other part of the subject's body that receives a chemical stimulation may improve the comfort and effect of the apparatus and method. In some methods of enhancing sleep as described herein, positioning step may include contacting skin of the forehead, hand or foot of the subject with the bioactive agent.

In some variations, an applicator is a topical or transdermal delivery device, such as a topical or transdermal patch with a stimulation chemical and configured to be placed or worn on a subject's body to provide stimulation to or across the subject's skin. An applicator may be size or shaped to fit onto a subject's skin, such as on forehead skin, hand skin, foot skin, toe skin, finger skin, etc. A topical or transdermal delivery device may have a stimulation transfer region. A topical or transdermal delivery device may deliver a stimulation chemical passively (e.g., passive diffusion) or actively (e.g., with a skin permeation enhancer, etc.). In some methods of enhancing sleep as described herein, the applicator comprises a patch comprising bioactive agent, the patch configured to be placed on the subject's skin a positioning step may include placing the patch on the subject's skin. A device such as a topical or transdermal delivery device may include an adhesive for adhering the device to the subject.

In general, a method as described herein includes delivering a bioactive agent on the subject's skin without requiring changing the subject's body temperature (skin temperature) by actually heating or cooling the subject's skin. For example, subject's body temperature (e.g., skin temperature) may not be detectably changed by a bioactive agent, or may be changed less than 1.0° F., less than 0.5° F., less than 0.2° F., etc. In some variations, the applicator is configured to remain at a passive temperature, and does not apply heating or cooling to the skin. A subject's body temperature may be changed while performing a method as described herein, however, this temperature change may be incidental and unrelated to the method of enhancing sleep, such as by another agent (e.g., an NSAID, another fever reducing drug, etc.) or by a physiological change in the body (e.g., due to normal body temperature variation due to sleep or enhanced sleep, etc.). Further, although the applicator may cover the skin (e.g., preventing normal cooling of the region under applicator), potentially raising the temperature of the skin, this may be considered a passive change in temperature and does not cause the therapeutic effects described herein. Further, in some variations the applicator may be configured to prevent this passive heating, by, for example, including pores or ventilation in the apparatus, and/or by constructing the apparatus from materials that readily permit the transfer of heat.

The methods described herein may be used for enhancing sleeping by delivering a bioactive agent to the subject's skin when the subject's temperature is above normal (e.g., fevered), and/or below normal, but in general these methods may be applied to a subject whose body temperature (skin temp) is in a normal range (e.g., 98.6° F.). In some variations the methods and apparatuses described herein may work best when the subject's skin temperature is within a normal or average range (or is considered “normal” for the individual. For example, the methods described herein may be applied to a subject's whose body (e.g., skin) temperature is less than 102.5° F., less than 101.5, less than 100.5, less than 99.5, less than 99.0, etc.

In some variations, the system includes a disposable component and a reusable component. For example, the applicator may generally be reusable, but the skin-contacting (interface) portion of the applicator may be configured to be used once or a few times and then replaced. Thus, the apparatus may include a disposable interface. Methods of treatment described herein may therefore include a step of placing a disposable interface on the applicator before positioning the applicator on the subject, wherein the disposable interface forms at least a part of the stimulation transfer region and is configured to contact the patient's forehead. The disposable interface may cover all or part of the applicator, or it may have an adhesive or other securement to hold it to the applicator so that it contacts the skin.

As mentioned, the step of positioning the applicator may include positioning the stimulation transfer region so that the stimulation transfer region does not contact the top or back of the subject's head. The step of positioning the applicator may comprise positioning the stimulation transfer region only against the subject's forehead.

Also described herein is a method of enhancing sleep in a subject comprising positioning on a subject's skin an applicator having a bioactive agent configured to change the subject's sensation of temperature without the bioactive agent substantially changing the subject's skin temperature; delivering the bioactive agent to and/or across the subject's skin; changing the subject's sensation of temperature without the bioactive agent substantially changing the subject's temperature; and enhancing with the bioactive agent the subject's sleep.

Also described herein is a method of enhancing sleep in a subject having normal body temperature comprising: positioning on the skin of a subject having normal body temperature an applicator comprising a bioactive agent configured to stimulate (e.g., activate or inhibit) a thermal sensitive receptor; delivering the bioactive agent across the subject's skin; chemically stimulating the thermal sensitive receptor with the bioactive agent and creating a biological response with the stimulated thermal sensitive receptor; and enhancing the subject's sleep due to the activated and/or inhibited thermal sensitive receptor.

Also described herein is a method of enhancing sleep in a subject comprising: positioning on the skin of a subject's hand or foot an applicator having a bioactive agent configured to chemically stimulate a thermal sensitive receptor without the bioactive agent configured to substantially change a temperature of the subject; delivering the bioactive agent to and/or into the skin of the subject's hand or foot; and causing the thermoreceptors to signal as a result of the bioactive agent thereby enhancing the subject's sleep without substantially changing the subject's temperature (e.g., skin temperature).

In some variations, maintaining the stimulation of the transfer region comprises maintaining the stimulation for at least about 5 minutes, 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, for at least about 4 hours, and/or at a target stimulation level for the subject's entire sleep period.

Also described herein are methods of enhancing sleep in a patient, the method comprising: placing a disposable interface on an applicator, wherein the disposable interface forms at least a part of a stimulation transfer region of the applicator and is configured to contact the patient's forehead, hand, or foot; placing the transfer region of the applicator against the subject's forehead; and maintaining the stimulation of the transfer region at a target stimulation for at least a predetermined amount of time (for example, at least 15 minutes, at least 20 minutes, at least 30 minutes, at least 40 minutes, at least 45 minutes, at least one hour, at least 1.5 hours, at least 2 hours, at least 2.5 hours, at least 3.0 hours, etc.). The device may enhance bioactive agent delivery rate in the early stage after application to the skin.

In some variations, the method includes: positioning an applicator having a stimulation transfer region so that the stimulation transfer region overlies thermoreceptors in the subject's forehead, wherein the transfer region does not contact the periorbital or cheek regions of the subject's face; and maintaining the stimulation of the transfer region at a target stimulation while the subject is sleeping to enhance the subject's sleep.

Also described herein are methods of decreasing sleep onset latency, the method comprising: positioning an applicator on the forehead of an awake subject so that a stimulation transfer region of the applicator contacts the subject's forehead, wherein the stimulation transfer region does not contact the periorbital, or cheek regions of the subject's face; preparing the patient to sleep; and reducing sleep onset latency by maintaining the stimulation of the stimulation transfer region for at least 30 minutes.

As described above, maintaining the stimulation of the stimulation transfer region may comprise maintaining the stimulation for at least about at least 15 minutes, at least 20 minutes, at least 30 minutes, at least 40 minutes, at least 45 minutes, at least one hour, at least 1.5 hours, at least 2 hours, at least 2.5 hours, at least 3.0 hours, etc., and/or for the subject's entire sleep period.

Also described herein are methods of decreasing sleep onset latency, the method comprising: placing a disposable interface on an applicator, wherein the disposable interface forms at least a part of a transfer region of the applicator and is configured to contact the patient's forehead; positioning an applicator on the forehead of an awake subject so that the stimulation transfer region contacts the subject's forehead; and reducing sleep onset latency by maintaining the stimulation of the transfer region at a target stimulation for at least at least 15 minutes, at least 20 minutes, at least 30 minutes, at least 40 minutes, at least 45 minutes, at least one hour, at least 1.5 hours, at least 2 hours, at least 2.5 hours, at least 3.0 hours, etc.

For example, described herein are methods of enhancing sleep in a subject comprising: positioning an applicator having a bioactive agent that activates cold-sensitive, warm-sensitive, or warm-sensitive and cold-sensitive receptors on the skin of the subject's forehead; delivering the bioactive agent onto the subject's skin; and chemically inducing a sensation of warming or cooling on the subject's skin without heating or cooling to the subject's skin to reduce sleep onset, improve sleep maintenance, increase sleep duration, or increasing the ratio of deep sleep relative to light sleep in the subject.

As mentioned above and described in greater detail below, chemically inducing a sensation of warming or cooling on the subject's skin without heating or cooling to the subject's skin to reduce sleep onset typically includes chemically activating (e.g., by application and/or delivery of one or more bioactive agents) thermoreceptors in the subject's skin. The methods and systems described herein typically rely on the chemical and not thermal activation of the thermoreceptors, thus the applicator may not actively regulate, apply or modify the temperature when applying the chemical induction. Although indirect or passive changes in temperature on the skin may occur (e.g., due to the physical insulation of the skin by the applicator), and indirect changes in the skin temperature as a result of the body's thermoregulation (e.g., sweating in response to the applied chemical activation), the apparatuses and methods described herein typically do not directly modulate the temperature of the skin. However, any of the methods and apparatuses described herein may also include the application (or be configured to include the application) of thermal energy (heating/cooling) as well as the chemical activation described herein.

Also described herein are methods of increasing sleep duration, the method comprising: positioning an applicator on the forehead of a subject so that a transfer region of the applicator contacts the subject's forehead, wherein the stimulation transfer region does not contact the periorbital, or cheek regions of the subject's face; and increasing sleep duration by maintaining the stimulation of the transfer region for at least 1 hour while the subject is sleeping. In some variations, the method further comprises placing a disposable interface on the applicator before positioning the applicator, wherein the disposable interface forms at least a part of the transfer region and is configured to contact the patient's forehead.

Apparatuses capable and/or configured to perform these methods are also described. For example, an apparatus for enhancing sleep by decreasing sleep onset latency, increasing sleep duration, and/or increasing the duration of deeper sleep stages relative to stage 1 sleep, may include: an applicator configured to be worn over a subject's forehead, the applicator comprising a transfer region configured to contact the subject's forehead but not to the perioribtal, or cheek regions of the subject's face. The applicator may contain any number of bioactive agents or chemical compounds, such as capable of inducing either a cold sensitive or warm sensitive skin receptor response. One example of a bioactive agent that activates a cold sensitive skin receptor response is menthol and an example of a bioactive agent that activates warm sensitive skin receptors is capsaicin. In some embodiments, a compound may be placed directly on the intended site as a colloid, cream, gel, emulsion, liquid, ointment, lotion, paste, semi-liquid, solution, and solid. The amount of the active agent can be varied to achieve the desired amount of activation of the targeted skin receptor. Additionally, depending upon the length of time a bioactive agent remains effective, a dispensing device could be incorporated to maintain a targeted activation level. Furthermore, a response to a bioactive agent such as for triggering cold sensitive receptors could be further enhanced, such as by the addition of a compound or device configured to reduce the skin temperature, such as an evaporative hydrogel, etc. A response to a bioactive agent could be further enhanced by the addition of a compound or device configured to increase the skin temperature, such as by a thermal insulation of the bioactive agent that raises the skin temperature or addition of a substance that chemically generates heat. In some variations, the apparatus is configured so that the stimulation transfer region only contacts the forehead.

Also described herein are kits for enhancing sleep in a patient comprising: an applicator configured to be placed on the skin of subject and to transfer a bioactive agent configured to change a subject's sensation of temperature across the skin of the subject; and an instruction for use for placing the applicator on the skin for enhancing sleep in the subject. In some variations, applicator includes a topical patch having the bioactive agent. In some variations, the instruction for use further includes an instruction for placing the applicator on a forehead of a user having a normal body temperature. In some variations, the instruction for use further includes an instruction for placing the applicator on a forehead of a user. In some variations, an instruction for use further comprises an instruction for placing the applicator on a hand or foot of a user.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a top view of one example of an apparatus for enhancing sleep by chemical activation of temperature receptors.

FIG. 2 is a side sectional view of the apparatus of FIG. 1.

FIG. 3 is a top view of another variation of an apparatus for enhancing sleep by chemical activation of temperature receptors.

FIG. 4 is a side sectional view of another variation of an apparatus for enhancing sleep by chemical activation of temperature receptors.

FIG. 5 is an illustration of one variation of a method for enhancing sleep by chemical activation of temperature receptors.

FIG. 6 is a side sectional view of another variation of an apparatus for enhancing sleep by chemical activation of temperature receptors configured for electrotransport of one or more bioactive agents (that chemically activate temperature receptors).

FIG. 7 is a top view of the variation shown in FIG. 6.

FIG. 8 is an example of another variation of an apparatus for enhancing sleep that includes a forehead applicator. In this variation, the forehead applicator is coupled (wirelessly or by wires) to a controller (stimulation device control unit) to control the operation of the forehead applicator.

FIG. 9 is a section side view of another variation of a forehead applicator.

FIG. 10 illustrates another variation of a forehead applicator as described herein. In this variation, rather than, or in addition to, applying chemical and/or thermal energy (heating/cooling), the apparatus applies one or more of mechanical (e.g., pressure, vibration, etc.), electrical, and/or magnetic stimulation.

### DETAILED DESCRIPTION

Described herein are apparatuses (including devices, kits, and systems) and methods that modulate sleep. Specifically, in some variations, these described include apparatuses and methods that provide chemical (rather than thermally) activation of thermoreceptors on the subject's body to modulate sleep. For example, described herein are apparatuses and methods that chemically stimulate thermoreceptors on the subject's skin (e.g., forehead, hands, and/or feet) for a period of time to induce a sensation of temperature (heat or cold) without significantly altering the person's skin temperature to improve sleep quality, including reducing sleep-onset latency, enhancing depth of sleep, and/or extending the amount of time a subject sleeps. In some variations the subject may be a subject suffering from insomnia or another sleep disorder. Also described are apparatuses that alternatively or additionally apply one or more of mechanical (e.g., vibrational, pressure, etc.), electrical and/or magnetic stimulation to the skin of a subject's forehead to modulate sleep.

The restorative nature of sleep and studies described in the medical literature demonstrating abnormal hyperarousal in insomnia patients suggests that the restorative aspects of sleep can be linked with a heteromodal association cortex, a region of the brain that receives input from multiple sensory and/or areas, especially in the frontal regions. Two studies were performed to understand the regional cerebral metabolic correlates of this. In the first study, changes in regional cerebral metabolism that occur between waking and sleep in healthy subjects were identified. In the first study, fourteen healthy subjects (age range 21 to 49; 10 women and 4 men) underwent sleep studies with concurrent EEG (electroencephalography) to measure changes in electrical activity in the brain and [18F]fluoro-2-deoxy-D-glucose ([18F]-FDG) positron emission tomography (PET) scans to image changes chemical activity in the brain during waking and NREM sleep. The first study showed that whole brain glucose metabolism declined significantly from waking to NREM sleep in healthy subjects. Relative decreases in regional metabolism from waking to NREM sleep were found in particular areas of the brain: heteromodal frontal, parietal and temporal cortex, and in dorsomedial and anterior thalamus. These findings are consistent with a restorative role for NREM sleep largely in cortex that subserves essential executive function in waking conscious behavior. In the second study, changes in regional cerebral metabolism were identified that occur between usual NREM sleep and recovery NREM sleep following a night of sleep deprivation. In this study, homeostatic sleep need, or sleep drive, was modulated in a within-subjects design via sleep deprivation. Four young adult healthy male subjects (mean age+s.d.=24.9±1.2 years) received NREM sleep using [18F]fluoro-2-deoxy-D-glucose positron emission tomography ([18F]-FDG PET) assessments after a normal night of sleep and again after 36 hours of sleep deprivation. Both absolute and relative regional cerebral glucose metabolic data were obtained and analyzed. In relation to baseline NREM sleep, subjects' recovery NREM sleep was associated with 1) increased slow wave activity (an electrophysiological marker of sleep drive); 2) global reductions in whole brain metabolism; and 3) relative reductions in glucose metabolism in broad regions of frontal cortex, with some extension into parietal and temporal cortex. The results from the second study demonstrate that the homeostatic recovery function of sleep following sleep deprivation is associated with global reductions in whole brain metabolism as well as greater relative metabolic reductions in broad regions of largely frontal, and related parietal and temporal cortex. In other words, sleep deprivation was shown to accentuate the normal decrease in brain metabolism observed during NREM sleep. A medical device or bioactive agent that alters metabolism in a pattern similar to that seen during healthy sleep or recovery sleep following sleep deprivation may therefore benefit patients having a sleeping disorder, including in particular insomnia patients.

A study of insomnia patients investigated how these normal changes in brain metabolism become disturbed in insomnia patients. Insomnia patients and healthy subjects completed regional cerebral glucose metabolic assessments during both waking and NREM sleep using [18F]fluoro-2-deoxy-D-glucose positron emission tomography (PET) scans. Insomnia patients showed increased global cerebral glucose metabolism during sleep and wakefulness. A group x state interaction analysis confirmed that insomnia subjects showed a smaller decrease than did healthy subjects in relative metabolism in going from waking to NREM sleep in the certain areas of the brain (ascending reticular activating system, hypothalamus, thalamus, insular cortex, amygdala and hippocampus and in the anterior cingulate and medial prefrontal cortices). While awake, in relation to healthy subjects, insomnia subjects showed relative hypometabolism in a broad region of the frontal cortex bilaterally, left hemispheric superior temporal, parietal and occipital cortices, the thalamus, hypothalamus and brainstem reticular formation. This study demonstrated that subjectively disturbed sleep in insomnia patients is associated with increased brain metabolism. The inability of these insomniacs to fall asleep may be related to a failure of their arousal mechanisms to decrease activity when going from waking to sleep. Further, insomniac's daytime fatigue may reflect decreased activity in prefrontal cortex that results from inefficient sleep. These findings suggest there are interacting neural networks involved in the neurobiology of insomnia. These networks include a general arousal system (ascending reticular formation and hypothalamus), an emotion regulating system (hippocampus, amygdala and anterior cingulate cortex), and a cognitive system (prefrontal cortex). Notably, ascending arousal networks are functionally connected to cortical regions involved in cognitive arousal at the cortical level which can feedback and modulate more primitive brainstem and hypothalamic arousal centers. A medical device that alters metabolism in one or more portions of this network could benefit insomnia patients and produce more restful sleep.

A second study in insomnia patients was conducted to clarify the cerebral metabolic correlates of wakefulness after sleep onset (WASO) in primary insomnia patients testing the hypothesis that insomnia subjects with more WASO would demonstrate increased relative metabolism especially in the prefrontal cortex given the role of this region of the brain in restorative sleep and in cognitive arousal. Fifteen patients who met DSM-IV criteria for primary insomnia completed 1-week sleep diary (subjective) and polysomnographic (objective) assessments of WASO and regional cerebral glucose metabolic assessments during NREM sleep using [18F]fluoro-2-deoxy-D-glucose positron emission tomography (PET). Both subjective and objective WASO positively correlated with NREM sleep-related cerebral glucose metabolism in the pontine tegmentum and in thalamocortical networks in a frontal, anterior temporal, and anterior cingulate distribution. These effects may result from increased activity in arousal systems during sleep and/or to activity in higher order cognitive processes related to goal-directed behavior, conflict monitoring, emotional awareness, anxiety and fear. These processes are thought to be regulated by activity of the prefrontal cortex.

Research to date has not conclusively identified the mode of action that temperature and temperature changes have on sleep. Based upon the existing research, the inventors have hypothesized that triggering either the warm or cold sensitive receptors in the skin may enhance sleep. Interestingly, a warm-sensitive or cold-sensitive receptor can be activated by warm or cold temperature applications to the skin or can be activated by chemical or other means (including topical application of a bioactive/chemical agent) without the direct contact of differential temperature. While not holding to a particular mechanism, it is believed that the thermal sensitive receptors respond in a similar fashion when activated regardless of the means of activation.

Described herein are methods and apparatuses (including devices, kits and systems) configured to provide chemical (rather than thermal) stimulation to a subset of thermoreceptors on the body (e.g., on a region of the subject's head or extremities) to modulate the subject's sleep. This may provide a non-thermal stimulation to activate or inhibit a thermal responsive pathway in the subject for enhancing the subject's sleep without requiring or (necessarily) causing a change in the subject's skin temperature. These agents may be topically applied either passively (e.g., onto the skin) or actively (e.g., driven by an electrical gradient), and may act through thermoreceptors in the skin to signal to the subject's brain to modulate (e.g., change, enhance, improve, etc.) the subject's sleep. Although not bound by a particular method of operation, these compositions, methods, and apparatuses are thought to work by affecting warm- and/or cold-sensitive thermoreceptors (neurons) in the body in communication with, e.g., the preoptic area and anterior hypothalamus of the brain, which may be sleep promoting to modulate the subject's sleep. These methods and apparatuses may function by first stimulating (e.g., activating and/or inhibiting) receptors (e.g., warm sensitive and/or cold sensitive receptors) in the skin that provides input to warm and/or cold sensitive neurons in the hypothalamus of the brain. They may do so without requiring or actually causing a change in temperature in the skin or the hypothalamus although they may create a sensation of temperature change that is experienced by the subject (e.g., due to stimulation or inhibition of the warm sensitive and/or cold sensitive neurons). They may also function to stimulate (e.g., activate or inhibit) other neurons, such as thermoinsensitive interneurons in pathways that are interlinked with warm or cold sensitive neurons or neural pathways. A bioactive agent or composition as described herein may be configured to promote (enhance) a subject's sleep without increasing the subject's body temperature, which may be normal body temperature.

In general, a bioactive agent as described herein may refer to a substance or combination of substances intended to furnish pharmacological activity (e.g., to have biochemical and/or physiological action on a cell, tissue, subject, etc.) and specifically to result in stimulation of thermoreceptors on the subject's skin. A bioactive agent may be or may include a chemical agent and may be a biologic agent (e.g., a genetically engineered protein). A bioactive agent may be naturally occurring or may be synthetic. A bioactive agent may activate or inhibit a receptor (thermoreceptor), and so may be a receptor agonist or a receptor antagonist. A bioactive agent may act in conjunction with other agents. A bioactive agent generally stimulates (binds to or enhancing binding to) a receptor (a biologic molecule including but not limited to a protein) in the subject's body to produce a measureable response. For example, a bioactive agent may bind to a sub-type of ligand-gated ion channels that cross the cell membrane of a cell, and the receptor, upon binding of the bioactive agent to a portion of the receptor outside a cell, may transduce a response inside the cell (intracellular response). A cell having a ligand-gated ion channel responsive to a bioactive agent may be a neuron and/or may stimulate a neuron. These bioactive agents may be referred to a chemical agents that activate thermoreceptors in the skin, and may typically act by topical and non-invasive application (e.g., by being applied to the skin either actively or passively).

Examples of bioactive agents are provided in detail below, but may generally include thermoreceptor agonists or antagonists, including warm receptor agonists. For example, a bioactive agent may include one or more of: 2-arachidonoylglycerol, 5(S)-HETE, 12(s)-HpETE, 15(S)-HpETE, arachidonoylethanolamide (AEA or anandamide), capsaicin, a capsaicinoid, a cold receptor agonist, icilin (1-(2-hydroxyphenyl)-4-(3-nitrophenyl)-3,6-dihydropyrimidin-2-one), dihydrocapsaicin, eucalyptol, homoedihydrocapsaicin, leukotriene B4, menthol (d menthol, dl menthol, l menthol), a menthol analogue, nordihydrocapsaicin, N-arachidonoyldopamine (NADA), N-oleoyldopamine (OLDA), oleoylethanolamide (OEA), resiniferatoxin (RTX), a TRPA (transient receptor potential A) agonist, a TRPA1 agonist, a TRPA(transient receptor potential A) antagonist, a TRPA1 antagonist, a TRPM 2, 4, or 5 agonist, a TRPM 2, 4, or 5 (melastatin transient receptor potential 2, 4, or 5) antagonist, a TRPV1-4 (transient receptor potential vanilloid-1-4) agonist, a TRPV1-4 (transient receptor potential vanilloid-1-4) antagonist, a TRPM 2, 4, 5, or 8 (melastatin transient receptor potential 2, 4, 5, or 8) agonist, or menthol, a vanilloid, and vanillyamide of n-nonanoic acid (NVA or PAVA).

In general, the methods of enhancing sleep in a subject may be and may be preferentially applied to subject's having a normal body temperature (e.g., 98.6° F.±0.5° F.). The bioactive agent may be applied in a manner that is comfortable (and compatible with enhancing sleep) and may be applied in a manner so that that the bioactive agent(s) do not desensitize the thermal receptors on the subject's head. Any of the methods described herein may use an applicator to deliver the bioactive agent that chemically modulates the thermoreceptors. In general, an applicator may include a skin-contacting surface (e.g., gel, foam, reservoir, etc.) that includes one or more bioactive agents for delivery. The applicator may also include an attachment to secure the applicator to the proper region of the subject's body, such as the head (forehead), hands, feet, etc. For example, the applicator may include a strap, belt, and/or adhesive. The applicator may be configured to minimize temperature changes when applied (e.g., it may be porous, or may be constructed from materials that readily transfer thermal energy; however in some variations the apparatus is configured so that it is made of a material having a low thermal transfer coefficient). Examples of applicators are provided below and in the figures.

A method of modulating sleep as described herein may generally include: contacting the skin with a bioactive agent configured to modulate a cold sensitive and/or warm sensitive receptor on the skin; evoking a sensation of warm or cool with the bioactive agent without substantially changing the subject's skin temperature; and thereby enhancing the subject's sleep with the bioactive agent. The bioagent may be delivered either before or during sleep (e.g., before a desired sleep time, in order to enhance sleep). The bioagent may be applied by positioning an applicator for delivery of the bioagent on the subject's skin.

Although in general, the apparatuses and methods described herein are for use with subject's having a normal body temperature (e.g., approximately 98.6° F.), the bioactive agent or compositions described herein may be given to a subject who a higher than normal temperature (a fevered person) or a lower than normal temperature. In general it may be beneficial to use these methods with subjects having a temperature in the normal range (e.g., a non-fevered subject). Normal temperature may vary from individual to individual and through the course of the day for a given individual. For example, normal body temperature is often highest in the evening. Normal body temperature may be affected (increased or decreased) by various factors, such as medication, high ambient (room) temperature, being outdoors, physical activity, ovulation, etc. Children's average daily temperatures are generally higher than adults' average daily temperature; and so their normal temperature range is generally higher than adults' normal temperature. Normal temperature for an individual is their normal daily temperature (e.g., the temperature they have when they do not have a fever and/or when they are not sick, etc.) and this temperature may be affected (increased or decreased) by factors other than fever or health status. Thus, in some examples, a bioactive agent or composition may be given to a subject with an oral temperature between 97.6° F.-99.6° F., about 98.6° F. (+/−0.1, 0.2, 0.3. 0.4, 0.5, 0.6. 0.7, 0.8, 0.9 degrees F.) or equivalent in Celsius (e.g., 37.0° C.+/−0.1, 0.2, 0.3. 0.4, 0.5, 0.6. 0.7, 0.8, 0.9 degrees C.) or when the subject's temperature is below any of these temperatures or below 99, 99.1, 99.2, 99.3, 99.4, 99.5, 99.6, 99.7, 99.8, 99.9 or 100.0° F. or below 38.0° C. Body temperature may also or instead be determined or measured in other ways such e.g. axilla temperature, ear temperature, rectal temperature, skin temperature, etc. These determinations may be used to determine normal or abnormal temperature, include determining if the subject has a fever. In some cases, a bioactive agent or composition may be given to a subject who has a fever to enhance a fevered subject's sleep. A subject whose temperature is higher than normal (such as higher than listed above or as determined or measured for an individual based on the individual's body normal temperature) such as above 99.5° F., above 100° F., above 101° F., above 102° F., etc. or whose hypothalamic temperature set-point is at least 0.5 or 1.0° F. above normal generally has a fever and a bioactive agent or composition may be given to such subjects to enhance their sleep. A bioactive agent(s) or composition as described herein for enhancing sleep may be given to a subject in need of enhanced sleep. A subject receiving a bioactive agent may or may not have other conditions, symptoms, etc. that are being treated. Thus a bioactive agent(s) or composition may be given to a subject who is experiencing aches, fever, pain, etc. or to a subject who is free from or is not treating aches, fever, pain, etc. (e.g., is pain-free, is non-fevered, etc.).

In any of the methods described herein, one or more bioactive agent may be delivered onto (topically) a subject's skin. In some variations, one or more bioactive agents may be delivered into a subject's skin or body (e.g., using microneedles, etc.) to contact thermal sensitive receptors, and may bind to thermal sensitive receptors in the skin. A bioactive agent may be placed on or near the skin surface and moved across the skin (topical delivery) such as by active delivery, flowing passively, iontophoresis, etc. A bioactive agent may be placed inside the skin, such as by injection, etc.

An apparatus or method as described herein may produce a sensation of warming or cooling (temperature change) in a subject. A sensation of warming or cooling may be local/regional and generally in the area of the body to which the bioactive agent was applied or may be non-localized such as in the brain or throughout the body (systemic). For example, a locally applied bioactive agent may have a biological effect in a subject's brain through a signaling pathway such that a subject has a non-localized sensation of warming or cooling. As mentioned above, a bioactive agent may change a subject's sensation of temperature without the bioactive agent substantially changing the subject's normal body temperature. A bioactive agent or an apparatus to deliver a bioactive agent to a subject may cause essentially no or only a minimal (but not substantial) change in temperature. For example, a bioactive agent may cover a portion of a subject's skin and thus may cause no or a minimal (e.g., less than 0.1° F., less than 0.2° F., less than 0.3° F., less than 0.4° F., less than 0.5° F.) increase in skin temperature. Alternatively, in some variations an apparatus for delivering a bioactive agent may cause a small increase in skin temperature to at least a portion of a subject's body. For example, a patch or headband for delivering a bioactive agent may insulate a portion of a subject's skin and cause a localized (passive) temperature increase; this temperature increase is not typically attributable to the bioactive agent(s), but may be limited to the insulative effect of the apparatus. Additionally, while a bioactive agent as described herein for enhancing sleep generally stimulates (activates or inhibits) a thermally sensitive receptor and enhances sleep without causing or requiring a change in the subject's temperature, in some variations, a method or apparatus or a different agent used at the same time that the bioactive agent is enhancing sleep may change the subject's temperature.

Activating (or inhibiting) a thermally sensitive receptor in the skin of the subject's arm(s), foot/feet, forehead (e.g., scalp), leg(s), torso etc. with a bioactive agent as described herein may improve sleep in an insomnia or other sleep-troubled subject, allowing them to more easily and quickly transition to sleep and to subsequently obtain more restful sleep during their sleep time (during the night). A bioactive agent may be placed on a subject's forehead, foot or part of a foot, or hand or part of a hand. A subject treated as described herein may be suffering from acute insomnia (e.g., brief insomnia or insomnia that is not chronic) or chronic insomnia (disrupted sleep that occurs at least three sleep periods (nights) per week and lasts at least three months). An increase in slow wave sleep may be expected to lead to reductions in metabolic activity in frontal cortex otherwise seen in insomnia patients when treated using the compositions, methods, and apparatus described herein.

In some variations, a method of enhancing sleep includes positioning on the skin of a subject's hand or foot an applicator having a bioactive agent configured to stimulate a thermal sensitive receptor without the bioactive agent configured to substantially change a temperature of the subject; delivering the bioactive agent into the subject's hand or foot; and chemically stimulating with the bioactive agent the thermal sensitive receptor thereby enhancing the subject's sleep without the bioactive agent substantially changing a temperature of the subject.

As mentioned above, a composition for enhancing sleep may include a bioactive agent configured to stimulate (activate or inhibit) a thermoreceptor (e.g. a cold sensitive receptor, a warm sensitive, a thermoinsensitive receptor, e.g., in an interneuron in a cold or warm sensitive pathways interlinked with a thermal sensitive receptor) in the skin, hypothalamus or other part of the body that provide input to warm and/or cold sensitive neurons in the hypothalamus of the brain. A bioactive agent may create a biological response by stimulation of thermal sensitive neurons in the hypothalamus and thereby enhance sleep. A composition for enhancing sleep may do so without requiring (or causing) essentially any or a substantial change in a body temperature (e.g. axilla temperature, ear temperature, oral temperature, rectal temperature, skin temperature, etc.). (Such compositions may paradoxically also or instead signal other receptors such as temperature insensitive or nociceptors that sense pain). Thus, a bioactive agent or composition may behave as a receptor agonist or antagonist to selectively activate or inhibit a thermal sensitive receptor (thermoreceptor). A bioactive agent may inhibit or activate a warm-sensitive receptor, a cold-sensitive receptor, and/or an interneuron in one of these pathways. A bioactive agent for enhancing sleep as described herein may include 2-arachidonoylglycerol, 5(S)-HETE, 12(s)-HpETE, 15(S)-HpETE, arachidonoylethanolamide (AEA or anandamide), capsaicin, a capsaicinoid, a cold receptor agonist, icilin (1-(2-hydroxyphenyl)-4-(3-nitrophenyl)-3,6-dihydropyrimidin-2-one), dihydrocapsaicin, eucalyptol, homoedihydrocapsaicin, leukotriene B₄, menthol (d menthol, dl menthol, l menthol), a menthol analogue, nordihydrocapsaicin, N-arachidonoyldopamine (NADA), N-oleoyldopamine (OLDA), oleoylethanolamide (OEA), resiniferatoxin (RTX), a TRPA (transient receptor potential A) (e.g., TRPA1) agonist, a TRPA(transient receptor potential A) (e.g., TRPA1) antagonist, a TRPM 2, 4, or 5 agonist, a TRPM 2, 4, or 5 (melastatin transient receptor potential 2, 4, or 5) antagonist, a TRPV1-4 (transient receptor potential vanilloid-1-4) agonist, a TRPV1-4 (transient receptor potential vanilloid-1-4) antagonist, a TRPM 2, 4, 5, or 8 (melastatin transient receptor potential 2, 4, 5, or 8) agonist, or menthol, a vanilloid, vanillyamide of n-nonanoic acid (NVA or PAVA), a warm receptor agonist, etc. Capsaicin is a chemical found naturally in different types of peppers, such as cayenne, habanero, and tobasco chilies, and is linked to the sensations of heat that are experienced when Chile peppers are eaten or placed on other sensitive areas of the skin, especially on mucous membranes. Capsaicin also affects pain reception (nociception) in the body, and can both cause and reduce pain. It is sometimes given topically (in products such as Arthicare, Capsagel, Capzasin-P, Zostrix, and Zostrix-HP) as a painkiller for temporary relief of aches and pains such as from diabetic neuropathy (nerve pain), psoriasis, osteoarthritis, and rheumatoid arthritis. Before capsaicin acts to reduces pain, it acts initially to increase sensations of pain and burning, and so may a numbing agent may be used with capsaicin to control these initial sensations of pain from the capsaicin and allow the capsaicin to act to reduce pain over a longer time period. Exactly how capsaicin acts to relieve pain is not known, but it has been suggested that it may act on or “defunctionalize” slower acting pain receptors and preventing these pain receptors from reporting pain. Menthol chemically triggers cold-sensitive (TRPM8) receptors, such as those in the skin. Menthol is an organic compound and can be synthesized or obtained naturally from the Mentha arvensis (mint) plant. Menthol is very quickly broken down in the blood. In some examples, a composition includes capsaicin, menthol, or another active (e.g., sleep improving agent) at a concentration up to about 20%, up to about 10%, up to about 8% and/or greater than 0.5%, 1%, 2%, 3%, 4%, 5%, 6%, 7%, 8% ((w/w or weight of bioactive agent/weight of solution)×100%). A solution may be an aqueous solution, a gel, etc. A composition as described herein may be especially useful when applied in a patch.

A composition of one or more bioactive agents as described herein may have or be part of any form, such a colloid, a cream, a gel, an emulsion, a liquid, a lotion, a paste, an ointment, a semi-liquid, a solution, a solid, etc. A composition may include another substance(s), including but not limited to an anti-oxidant agent, an emulsifying agent, a free radical scavenger, lubricant, lubricity agent, moisturizer, numbing agent, an oil, pH buffering agent, a polymer, a preservative, silicone oil, sodium chloride, sodium hyaluronate, a solubilizing agent (e.g., alcohol, acetone, ethyl acetate), urea, viscosity control agent, vitamin, vitamin analog Vitamin B, Vitamin E (tocopherol), water, etc. A composition may be pharmaceutical grade and may include pharmaceutically acceptable additives. A bioactive agent for improving sleep may be applied from any container or device that contains or can deliver the agent (e.g., an applicator, an apparatus, a bottle, a film, a jar, a patch (e.g., a transdermal patch), a pump, a pump bottle, a stick, a tub, a tube, a wipe, etc.).

In some variations, one or more bioactive agents to improve sleep may be delivered to a subject using a membrane for bioactive agent (sleep enhancing agent) delivery, such as by applying a membrane to a forehead, foot or part of a foot (e.g. a toe) or hand or part of a hand (e.g., a finger). A membrane may be a pliable structure and may deliver a bioactive agent for enhancing sleep to a subject's skin. In some variations, a membrane may be a selectively permeable structure and may allow some materials to pass through and may prevent other materials from passing through. A membrane may have pores configured to control a delivery rate of a bioactive agent across the membrane. A membrane may be part of a topical or transdermal delivery system configured to deliver a bioactive agent to or across the skin of a subject. A topical delivery system delivers a bioactive agent to the skin, and the bioactive agent acts locally. The agent does not spread systematically throughout the body to have a direct effect elsewhere in the body. For example, a topically delivered bioactive agent may bind to and stimulate (inhibit and/or activate) thermal sensitive receptors (e.g., warm receptors or cold receptors) beneath or near skin where it was delivered. (It does not spread systematically throughout the body through the bloodstream). Once inhibited or activated however, a thermal sensitive receptor may send a signal that is carried or transmitted to other parts of the body, such as a bioactive agent as described herein sending a signal to the hypothalamus of the brain that controls sleep. The hypothalamus or other structures in the body that are signaled may then control other functions such as behavioral or physiological functions. A transdermal delivery system delivers an agent widespread through the body and to the bloodstream. A bioactive agent in the bloodstream may be delivered by the bloodstream to the body. A bioactive agent may act both topically and transdermally. A bioactive agent may be placed on a membrane or may be contained by a membrane, such as contained in a pouch or enclosed by a membrane. A membrane may be or may include a thin piece of a material, a layered material, a plurality of layers, a pouch, a layer overlying a reservoir of bioactive agent, etc. A membrane may be any shape that fits over a part of a body. In some examples, a membrane may sufficiently large to cover a portion of a subject's forehead (all, most, part, at least 75%, at least 50%, at least 25%, etc. of the surface area of the forehead). In some examples, a membrane may be shaped (e.g., contoured or cut) to fit around a part of the body, for example to fit around a thumb or part of a thumb, a finger or part of a finger, more than one finger, a back of a hand, a toe, more than one toe, a top of a foot, etc. In some examples, a membrane may sufficiently large to cover a portion of a subject's finger, toe, foot, or hand, such as most, part, at least 75%, at least 50%, at least 25%, etc. of the surface area of the finger, toe, foot, or hand or may only cover a portion such as less than 100%, less than 75%, less than 50%, less than 25%, etc.). or in between any of these sizes. A bioactive agent may be placed on a membrane or held by a membrane and may flow or diffuse across a membrane. Capsaicin, menthol or another bioactive agent may be placed on a membrane at any amount that improves sleep. For example, capsaicin, menthol, or another bioactive agent may be present at least 1 microgram (mcg), at least 5 mcg, at least 50 mcg, at least 100 mcg, at least 200 mcg, at least 300 mcg, at least 400 mcg, at least 500 mcg, at least 600 mcg, at least 700 mcg, at least 800 mcg, at least 900 mcg, or at least 1 gram per square centimeter (cm) of membrane or may be present below or between any of these amounts (e.g., more than 1 mcg and less than 50 mcg per square centimeter (cm) of membrane, between 600 mcg and 700 mcg per square centimeter (cm) of membrane, etc.

In some variations, a bioactive agent(s) for enhancing sleep may be delivered over a period of time. A bioactive agent may be configured for controlled delivery over time or its delivery may be controlled by the applicator. A bioactive agent in a subject's body is generally broken down (metabolized) by the body; and so the amount of bioactive agent available is reduced over time. The half-life of a bioactive agent is amount of time required for half of the initial amount of the bioactive agent in a subject to be eliminated or disintegrated (by the body). A bioactive agent with a short-half life may have a direct effect on the subject only for a very short time. As explained elsewhere, a bioactive agent may have an indirect effect on a subject after the bioactive agent is eliminated or disintegrated, due to neural or receptor desensitization or sensitization, etc. For example, once menthol reaches the blood, it is broken down almost immediately. The half-life of a bioactive agent that is applied to a subject (e.g., applied topically to the subject's skin) may depend on various factors, such as the type of skin to which an agent is applied, how quickly a bioactive agent crosses the skin barrier (epidermis), how quickly it reaches the blood stream, how quickly it reaches the liver, and is metabolized, etc. In order for menthol or another bioactive agent to have a direct effect on the body (e.g., warm sensitive or cold sensitive receptors or neurons) over a sleep period (e.g., more than minutes, more than an hour, more than two hours, etc.), it may be helpful to supply an additional quantity(ies) of one or more bioactive agents (e.g., menthol) to the subject over time. One or more bioactive agent(s) may be delivered (released) more or less continuously over at least 30 minutes, at least 1 hour, at least 2 hours, at least 3 hours, at least 4 hours, at least 5 hours, at least 6 hours, at least 7 hours, at least 8 hours, etc. Alternatively or additionally, one or more bioactive agents maybe released discretely at predetermined (e.g., every 5 minutes, every 10 minutes, every 15 minutes, every 20 minutes, every 25 minutes, every 30 minutes, every 35 minutes, every 40 minutes, every 45 minutes, every 50 minutes, every 55 minutes, every 60 minutes, etc.), at random intervals, or based on a sensed parameter (e.g., based on EMG, ECG, PSG, etc.). The amount of time a bioactive agent for enhancing sleep is delivered to a subject may be chosen for any reason. Activity due to a bioactive agent for enhancing sleep may only be desired for a limited amount of time. For example, activity may only be desired for a limited amount of time to allow the subject to wake up (or gradually come out of deep sleep) when it is wake time or close to wake time. In other examples, a subject may only need a sleep enhancing bioactive agent for part of a sleep cycle or sleep preparation time because only a portion of their sleep cycle is in need of help. For example, a subject may need help falling asleep and may use a sleep enhancing bioactive agent to help them fall asleep but may not need a sleep enhancing bioactive agent to keep them asleep. Bioactive agent delivery over time (over a sleep cycle) may be controlled by a bioactive agent delivery controller. Delivery may be controlled by controlling how quickly a bioactive agent is released from a delivery device, how quickly the bioactive agent diffuses across the skin, how quickly it is made available in the body, etc. For example, a membrane for topical delivery of a bioactive agent may have pores that control how quickly bioactive agent is released; a bioactive agent may be modified or coated to slow or prevent metabolism by the subject's body, etc. The effectiveness of a bioactive agent or a bioactive agent delivery controller for controlling delivery of a bioactive agent may be analyzed (e.g., assayed or inferred). For example, effectiveness may be analyzed by providing a subject a bioactive agent and assaying one or more parameters related to sleep quality, such as using performing a polysomnogram (PSG) using sensors (electrodes) to measure the amount of blood oxygen, blood pressure, brain activity (brain waves), chin muscle activity, eye movements (REM sleep/NREM sleep and sleep stage), effort exerted during breathing, heart rate and rhythm, leg movements, etc. during sleep. Effectiveness of a bioactive agent or bioactive agent delivery controller may be analyzed such as by the subject or an observer recording sleep quality events (in an app or journal). The subject may indicate (record) responses to the Epworth Sleepiness Scale, a widely accepted questionnaire used in sleep studies to assess daytime sleepiness as an indication of quality of sleep (at night) or using a Multiple Sleep Latency Test (MSLT) for daytime sleepiness, which is a test performed with sensors to determine brain activity and eye movements to determine how quickly a person falls asleep in a quiet environment during the day. The rate of movement of a bioactive agent out of a controller, such as out of a topical membrane patch, may be measured as an indication of how much bioactive agent may be delivered to an individual over time. As indicated above, a bioactive agent that acts on a thermal sensitive receptor and/or in a cold-sensitive or warm-sensitive pathway may cause a sensation of coolness or warmth. Effectiveness of a bioactive agent or a bioactive agent delivery controller may be analyzed using a subjective test (such as a subject indicating on a scale of 1-10 how cool or warm the subject feels over time). Capsaicin binds to and activates the transient receptor potential vanilloid 1 receptor in the body. This receptor is highly conserved across the animal kingdom (humans, rats, the laboratory model C. elegans nematode, the fruit fly Drosophila melanogaster, etc.) and a number of assays have been developed in animal models including that can be used for analyzing the effectiveness (or effective amount) of a bioactive agent or a bioactive agent available from a bioactive agent delivery controller.

Bioactive agent delivery profile may be varied over time. Varying its delivery profile may increase bioactive agent effectiveness, reduce unwanted side effects, etc. A bioactive agent may be delivered at a relatively higher level followed by delivery at a lower level (e.g., its delivery profile may be tapered) or may be delivered at a lower level followed by delivery at a higher level (e.g., its delivery profile may be ramped up). Tapering bioactive agent delivery may be useful for reducing the depth of sleep, such as to aid or allow a subject to awaken gradually, reducing side effects by delivering a lower dose, etc. Ramping bioactive agent delivery may be useful to allow improve sleep depth, etc.

In some variations, a bioactive agent(s) for enhancing sleep may be delivered or configured for delivery in amounts that change during the course of a sleep period or over two or more sleep periods. In some instances, a cold or warm receptor that responds to a bioactive agent may become sensitized (more responsive) in response to application of the bioactive agent and thus a smaller quantity of bioactive agent may be sufficient in subsequent doses to have a desired sleep enhancing effect. A first dose may be at a higher level than a second dose, etc. A body receptor may instead or additionally (e.g., before or after becoming sensitized) become desensitized (less responsive) in response to application of the bioactive agent and thus a larger quantity of bioactive agent may be given in subsequent doses to have a desired sleep enhancing effect. For example, the body has a complex response to capsaicin, and it has been reported that capsaicin causes an initial sensitization in the transient potential receptor vanilloidl in the pain nerves to which it binds, followed by a period of desensitization (with a decrease in pain sensation). A bioactive agent for enhancing sleep as described herein may causes an initial sensitization (e.g., by a warm or cold sensing receptor or neuron) followed by a period of desensitization and thus an amount of bioactive agent provided to a subject may be altered based on its expected effect on desensitization or sensitization or a warm or cold sensing receptor or neuron.

In some variations, a plurality of (e.g., two, three, four or more than four) bioactive agents for enhancing sleep may be delivered to a subject for enhancing sleep. Such agents may be delivered simultaneously (at the same time), at different times, at alternating times, before or during a single sleep cycle, before or during more than one sleep cycle, etc. Such plural delivery of bioactive agents may aid in causing or preventing desensitization, sensitization, toxicity, etc. It may be useful for delivering bioactive agents that act on different receptors or different nerves (act using different mechanisms), have different half-lives, are broken down by different enzymes, etc. or at different times. For example, it may be useful to deliver a first bioactive agent that works on a warm sensitive neuron and a second bioactive agent that works on a cold-sensitive neuron.

In general, any of the apparatuses for enhancing sleep may include an applicator (e.g., pad, etc.) that fits against part of a subject's body and can be worn before and/or during sleep. An applicator may be configured to fit against any portion of a subject's body, but most commonly is configured to fit against the subject's head (e.g., the forehead), hand (such as the back, side or front of the hand or a finger or any portion of these) or foot (such as the bottom, side or top of the foot or toe or any portion of these). The applicator may be placed on part of the body and may lie relatively flatly (e.g., on a forehead) or may be wrapped or partially wrapped around the body (e.g., around a finger or toe). An applicator may lie passively against the body or may be held in place on the body, e.g., for example it may be held against the body by an adhesive, an elastomeric material, a hydrogel, a hook-and-loop (e.g., Velcro®), a polymer, a strap, another type of headgear. The applicator may also or instead be held in place by a material that produces an adhesive effect such as hydrogel. In some variations an active compound or ingredient is combined in a hydrogel type material or other materials commonly used for drug delivery through the skin. Other variations may consist of gels or creams that can be applied to the forehead that contain the active compounds or ingredients. The applicator may contain a disposable component that is replaced at specified intervals to replenish the active compound.

In some variations, a device includes an applicator configured to transfer a bioactive agent to the skin of a subject, the applicator configured to conform to and wrap around a foot or hand of a subject; and the bioactive agent configured to change the subject's sensation of temperature with the bioactive agent without substantially changing the subject's normal body temperature with the bioactive agent.

A composition as described herein may be configured to be released upon application to the subject's body (e.g., immediately) or may be configured to be released over a period of time (extended release) or to be released at a later time (e.g. in the middle of the night or the middle of a sleep cycle). (The timing of release to a subject may be controlled by an apparatus). A composition thus may be released or available immediately at the time of application to a subject or its release or availability may be delayed. An immediate release may be useful for example, for helping a subject fall asleep. An extended release may be useful, for example, for releasing a bioactive agent over the course of a sleep cycle (e.g., during a night) to enhance sleep over a period of time.

Also described herein are methods of enhancing sleep in a subject including: positioning on a subject's skin an applicator having a bioactive agent configured to change the subject's sensation of temperature without the bioactive agent substantially changing the subject's temperature; delivering the bioactive agent across the subject's skin; and changing the subject's sensation of temperature without the bioactive agent substantially changing the subject's temperature; and enhancing with the bioactive agent the subject's sleep.

Also described herein are methods of enhancing sleep in a subject including: positioning on the skin of a subject having normal body temperature an applicator comprising a bioactive agent configured to chemically stimulate a thermal sensitive receptor; delivering the bioactive agent across the subject's skin; stimulating the thermal sensitive receptor with the bioactive agent and creating a biological response from the stimulation of the thermal sensitive receptor; and enhancing the subject's sleep due to the stimulation of the thermal sensitive receptor.

An applicator may be held in contact with the subject's head with a head gear system. In one variation of the headgear component, a series of adjustable straps are used to selectively adjust the contact pressure of the applicator to the user. Other variations of the headgear can be constructed with elastic type material or without adjustability. An elastic material may be useful for applying contact pressure to a thermal applicator. Other variations may utilize both features, i.e. adjustable straps and elastic materials. In some variations an applicator may be (permanently) integrated with a headgear and in other variations, an applicator can be removable from a headgear.

As mentioned, an applicator portion of a bioactive agent delivery apparatus generally includes a skin-contacting region configured to lie against the subject's skin (e.g., skin of the forehead, hand or part of a hand, finger or part of a finger, foot or part of a foot, toe or part of a toe, etc. The skin-contacting region generally includes a stimulation transfer region. A stimulation transfer region provides a stimulation, which may be an activating or inhibiting stimulation, to a thermosensitive receptor. Stimulation generally occurs from the stimulation transfer region, which may be any part of the body, but in general will be the subject's forehead, hand, foot, finger or toe. An applicator may be configured so that other regions of the subject's head or face are not in contact with the stimulation transfer region; thus stimulation may only be applied to the forehead but not to other regions such as the eye orbits, cheeks, neck, back of the head, hairline, etc. Thus, in some variations an applicator may contact or cover other regions of the body, in addition to the forehead or other part of the body being stimulated, but those regions may not include a stimulation transfer region, and thus may not receive stimulation. An applicator may generally be configured to enhance wearer comfort. For example, an applicator may have a relatively thin thickness (e.g., less than 5 cm, less than 2 cm, less than 1 cm, etc.), so that it can be comfortably worn while sleeping. An applicator may adjustably fit to a variety of patient head circumferences. Thus, in some variations, application of a stimulation transfer region to a portion of a subject's body (skin) may be used to enhance sleep. In some examples, a treatment region may be limited to the forehead, hand, foot, toe, or finger, as mentioned above. For example, in one variation, a single transfer region of an applicator is shaped to cover the region of the forehead that overlies glabrous (non-hairy) skin. The frontal cortex is thought to be uniquely important among body regions for providing thermoregulatory information to the hypothalamus given that it has the highest thermal sensitivity of body surfaces and has a neural and vascular supply that are specialized for this function. The forehead allows a convenient surface for placing a pad during sleep applications (e.g., to minimize disturbing the subject). Thus, an arrangement of an applicator configured to provide a stimulation between the applicator and the forehead may benefit sleep.

Although the discussion above provides a proposed basis for stimulation of thermoreceptors for enhancing sleep, the methods and apparatus described herein may operate effectively regardless of the correctness of the proposed basis. Further, other chemical or electrical methods for stimulating skin thermal receptors, such as on the forehead, hand, foot, finger, toe, etc.) are included herein. These methods could be utilized in the regions and manners provided for the purpose of improving sleep such as utilizing the underlying brain mechanisms as described herein by utilizing a different method of providing stimulation (e.g., activation or inhibition) in these regions. In general, the methods and apparatus may be used to facilitate or enhance sleep. For example, a device may be used on the scalp in the region over the area of the forehead to provide a thermoregulatory stimulation to warm or cold sensitive hypnogenic neurons of the hypothalamus thereby facilitating or enhancing sleep. A device may be used prior to sleep to aid in sleep onset. A device may be configured to communicate with the subject's head surface area over the region of the frontal cortex. A device may be used to improve sleep in at least, but not limited to, the following conditions: improving healthy sleep, improving sleep in insomnia patients; improving sleep in individuals who experience sleeplessness; improving sleep in a jet-lagged subject; improving sleep in a sleep-disordered subject; improving sleep in a subject with a circadian rhythm disorder; improving sleep in a subject with a shift work disorder (e.g., someone who rotates work shifts frequently or works at night); improving sleep in individuals with neuropsychiatric disorders such as, but not limited to, depression, mood disorders, anxiety disorders, substance abuse, post-traumatic stress disorder, psychotic disorders, manic-depressive illness and personality disorders and any neuropsychiatric patient who experiences sleeplessness; improving sleep in patients with pain, including chronic pain, and headaches, including cluster, headaches, migraine headaches; improving sleep in patients with sleeplessness or insomnia secondary to other medical disorders such as cardiac, endocrinological, and pulmonary disorders; improving sleep in patients with neurologic disorders where sleeplessness or insomnia occurs including but not limited to tinnitus. The apparatus and methods may be useful in a clinic, at a fire-station, in a hospital, at home, in a jet or other public transport, etc.

In general, a stimulation transfer pad or patch (e.g., containing a bioactive stimulation agent) could be applied prior to getting into bed (prior to “good night time”). In general good night time may refer to the intended time that a patient will (or would like to) fall asleep; this may be the time that a patient goes to bed, or some time thereafter. Thus, for example, an apparatus could be applied to the subject a few minutes before getting into bed, five (5) minutes before going to bed, ten (10) minutes before going to bed, 30 minutes before going to bed, 45 minutes before going to bed, 1 hour prior to getting in to bed, etc., to facilitate the sleep onset process. Neural transmission may occur within seconds, therefore applying the device closer to the time of getting in to bed may enhance an ability of the device to improve sleep onset. If effects only on sleep onset were desired, a device could be removed prior to the subject falling asleep. In some variations a subject may wear a device before getting to bed (e.g., between an hour to a few minutes before going to bed) and then remove the device immediately before going to bed. The subject may also wear (and use) the device during sleep. An apparatus (including a stimulation transfer region of a transfer pad) could be applied when or after a subject gets into bed, and may be worn throughout sleep cycle (e.g., a night of sleep) to facilitate sleep across a night of sleep. In some variations, an apparatus may be configured to vary the time course of chemical stimulation (with a bioactive agent) to the probability of NREM and REM sleep stage occurrences. Brain temperature, brain blood flow, and brain metabolism vary in characteristic ways during a sleep cycle (e.g., a night of sleep) and are dependent on the stage of sleep an individual is in, as well as the duration of time from the beginning of sleep. NREM sleep stages include lighter stage 1 sleep, deeper stage 2 sleep and deepest stages slow wave sleep with slow wave sleep predominating in the first half of the night. REM sleep occurs cyclically across a night, every 60-90 minutes with progressively longer and more intense REM periods occurring in the latter parts of the night. Brain temperature, blood flow and metabolism tend to lessen in deeper NREM sleep and increase in REM sleep. The degree to which these changes occur are thought to be functionally important for sleep. As discussed above, the apparatus described herein to apply a stimulation to a subject's forehead or other part of the body may facilitate the deepening of NREM sleep, such as by activating warm or cold sensitive neurons in the hypothalamus which may subsequently lead to increased slow wave sleep. A variable stimulation time course may apply more stimulation (e.g., more bioactive agent) to the subject's forehead earlier in the night when slow wave sleep tends to be maximal, with less stimulation towards the end of the night when REM sleep and natural brain warming would be occurring.

Some disorders, such as depression, have characteristic alterations in REM sleep. A stimulation applicator may have predictable effects on the occurrence of REM sleep. One method of treatment may include a variable bioactive stimulation transfer over the course of the night that is intended to target the occurrence of REM sleep in a therapeutic manner. Alterations in REM and NREM sleep can occur in a variety of neuropsychiatric disorders. The general principle of altering the stimulation of the transfer region of the applicator (which may be referred to herein as a “mask”) to facilitate or diminish discrete aspects of deep NREM sleep or REM sleep that are directly related to a specific disorder may have therapeutic utility specific to the disorder.

The apparatuses described herein may also include or to operate with one or more sensors (or sensing subsystems) configured to determine a subject's sleep state. Sensors or sensing subsystems may include EEG (electroencephalogram) sensors, motion sensors (detecting sleep motions to determine sleep state), and/or body temperature sensors, or other means for determining sleep as known to those of skill in the art; additional examples are provided above and below. In some examples, altering the stimulation properties of an applicator may have predictable effects on sleep physiology. Some examples include measuring changes in sleep physiology and incorporate changes into a feedback loop, providing changes in stimulation. In this manner, an apparatus controller may adjust the apparatus stimulation that is applied in real time to achieve a desired physiological effect. In some variations, variable stimulation with defined changes can be delivered across a period of use with the changes linked to feedback from changes in the physiology of the body across the period of use. For example, the following physiological measures may be monitored and stimulation adjusted in real time according to the level of the physiological measure: presence or absence of REM or NREM sleep (as assessed by any method of REM/NREM sleep assessment by someone skilled in the art) such as EEG frequency, Heart Rate Variability, Muscle Tone or other means; depth of slow wave sleep, as measured by EEG wave analysis or other means; degree of autonomic arousal as measured by HR variability or other means; galvanic skin response; skin temperature, such as at the skin on the head underneath the device, or on skin at some other portion of the head not underneath the device, or peripheral skin temperature, or core body temperature (measured internally or by some external means) or some combined measure assessing thermoregulation of the head and periphery, or core body to peripheral temperature measure.

As mentioned, a subject using device may, in some variations, modify a stimulation profile across a period of use, with or without such changes linked to feedback. For example, a control on the apparatus may allow a subject using an apparatus to adjust the stimulation with a bioactive agent (according to their immediate comfort and treatment needs, either up or down some small increments. In some variations of the apparatus (devices and systems) described herein, an individual can set their go to bed times and desired get out of bed times (and/or good night time), and a preprogrammed algorithm may input start and stop at those times and provide incremental adjustments (e.g., increases or decreases in active agent levels) to occur on a relative basis over this time period. Such automated time calculations could be implemented for any variable schedule of stimulation (stimulation or inhibition) rates across any defined period of time.

In some variations an applicator includes a part, such as a lining combined with a dermatologic product, such as a skin rejuvenating product for contacting over the course of a night. In some examples, a cream configured to hydrate the skin and/or apply a medicament to the skin may be used. In some variations a lining (e.g., an inner or other lining) can be refreshed on a nightly or less frequent basis that can benefit the skin when applied over the night of sleep. Thus, an applicator, and particularly a skin-contacting portion of an applicator may be configured to be disposable and/or replaceable daily (e.g., nightly), every other day, every week, etc.

Any of the variations of the apparatuses described herein may also be configured to record, store and/or transmit data about the operation of the device and/or the subject using the device. A subject, or in the clinical management of a patient, a healthcare provider may want to know certain parameters about a patient and/or device use (over time, over multiple nights, etc.) such that care can be adjusted or optimized. In some variations, memory (e.g., a memory card, a memory chip, etc.), may automatically record all or some parameters and store them for later display and/or transmission to an individual or healthcare provider. Further, in monitoring their own care, a device user may want to know certain parameters of the patient and/or device over a single use or over multiple nights of use such that care can be adjusted or optimized. An apparatus may be configured to display and/or transmit such information, e.g., for uploading to a computing device (computer, mobile or communications device, website, etc.). In some variations, information such as this could be transferred to a healthcare provider's office or some other central database via a phone, internet, wireless technology etc. where someone could review the information and provide recommended adjustments in the treatment accordingly.

Examples of information that may be stored include, but are limited to any of the parameters described elsewhere herein and/or: core body temperature, depth of sleep as assessed by NREM sleep, EEG power in discrete frequency bands, measures of autonomic variability, periods of activity and/or wakefulness across the night, REM sleep or other sleep staging, skin temperature, subjective measures of sleep depth/comfort/satisfaction, sleep duration, etc. Any of the apparatuses (devices and systems) described herein may be configured to operate (or include as part of their operation) a gradual increase/decrease of the stimulation (e.g., ‘ramping’) over a predetermined amount of time. For example, in some variations an apparatus may be configured to include an alarm-clock (or ‘wakeup’) feature in which, at some predetermined/user selected time, stimulation (activation or inhibition) from applicator is changed (e.g., increased or decreased) to a predetermined level to stimulate the subject to wake up.

In some variations, a kit for enhancing sleep in a patient includes: an applicator configured to be placed on the skin of subject and to transfer a bioactive agent configured to change a subject's sensation of temperature across the skin of the subject; and an instruction for use of the applicator placing the applicator on the skin for enhancing sleep in the subject. As described above, an applicator (in a kit) may include a topical or transdermal patch having the bioactive agent. An instruction for use may also or instead include an instruction for placing the applicator on a forehead of a user having a normal body temperature. An instruction for use may also or instead include placing the applicator on a forehead of a user. An instruction for use may also or instead include an instruction for placing the applicator on a hand or foot or part of a hand or foot of a subject.

### EXAMPLES

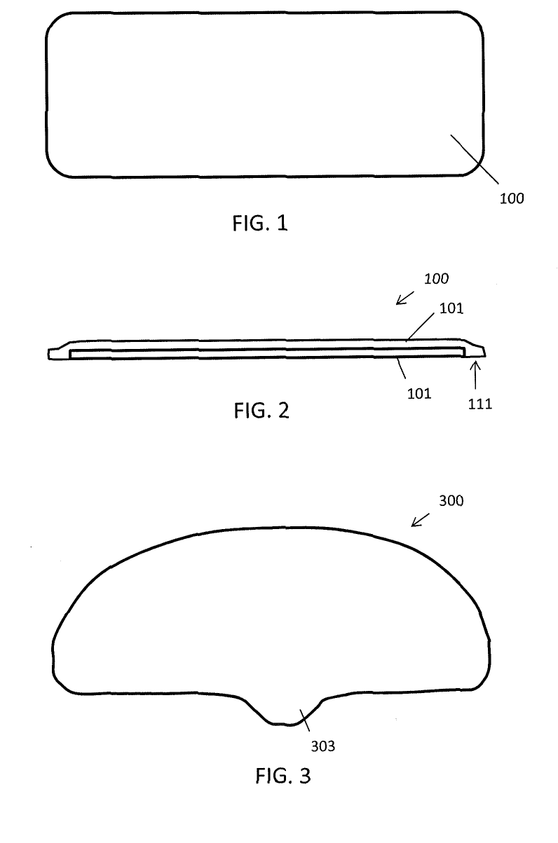

FIGS. 1-4 and 6-7 illustrate variations of apparatuses and FIG. 5 illustrates a variation of a method for modulating sleep by chemical activation of temperature receptors, as described above.

For example, FIGS. 1 and 2 show an exemplary apparatus configured as an adhesive patch that maybe applied and worn by a subject prior to falling asleep in order to chemically modulate thermoreceptors (warm- or cold-sensitive receptors) on the skin. In FIG. 1, the apparatus is configured as a patch that may be worn, for example, on a subject's hand, foot or forehead. The patch 100 may be adhesive, and may be secured on the skin so that a skin-contacting surface 101, visible in FIG. 2, is in communication with the skin. The skin-contacting surface 101 may be a reservoir, such as a gel (which may be adhesive) or pad that is typically loaded with (or contains) one or more bioactive agents that can be released onto the skin to activate (or in some variations inhibit) thermoreceptors on the skin to give the sensation of cooling or warming (depending on the combination of agents) without actually substantially changing the skin temperature. The gel may act as a reservoir of bioactive agent(s). This reservoir may be covered by the rest of the applicator body 101 which may protect and secure the reservoir region to the skin. The applicator body 101 may also include an adhesive surface 101; in FIG. 2, the adhesive surface surrounds the skin-contacting surface of the reservoir, and may include a biocompatible adhesive that allows it to be secured against the skin.

The applicator apparatus may be configured into a shape that is optimized for applying the topical bioactive agent to a particular body region. For example, FIG. 3 illustrates an example of a forehead applicator similar to that shown in FIG. 1. In this example the applicator 300 is shaped (on the outer perimeter of the patch body) to fit onto a forehead region (and may be oriented using the tab region 303. Any appropriate shape may be used, particularly shapes that mirror the skin regions onto which they are to be applied (e.g., the forehead, back of the hand, fingers, palm, wrist, bottom of the foot, top of the foot, toes, etc.).

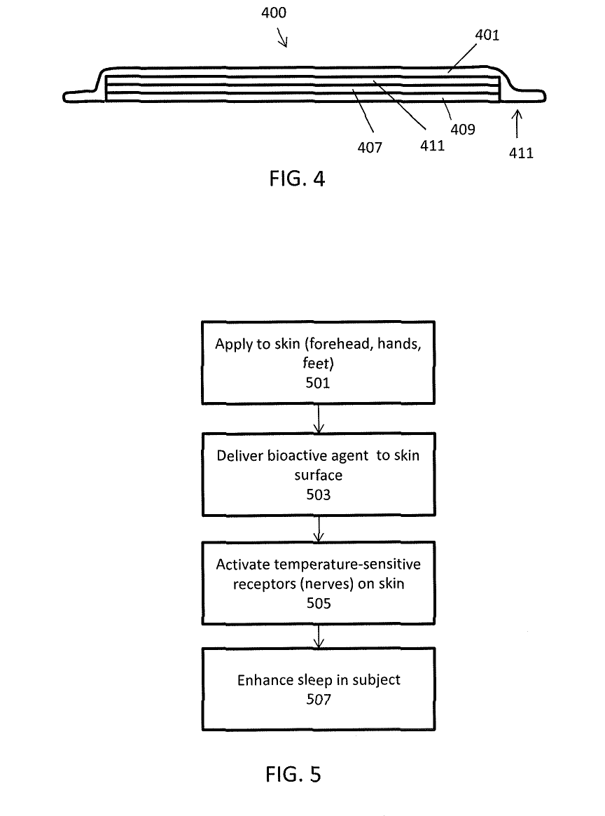

FIG. 4 illustrates another example of a section through an applicator apparatus such as the one shown in FIGS. 1 and 3. In this example, the apparatus include a plurality of reservoirs 407, 409, 411, which may each be loaded with one or more bioactive agents for delivery when the patch is applied to the skin. The configuration shown in FIG. 4 may be adapted in particular to provide different gradients of bioactive agents; distributing different bioactive agents in these different regions may allow controlled release (timed release) of the agents. For example, in some variation each layer may include a different bioactive agent that may be applied with a different time course. A first bioactive agent in the lowest (skin-contacting) layer 409 may be applied first, followed after a diffusion-dependent time by the bioactive agent in the second layer 407, and finally by the bioactive agent in the third layer 411. The device having different layers comprising different regions may be achieved by having each region initially isolated from each other (e.g., in the packaging) and then placed into contact with each other just before being applied to the skin (e.g., by the subject, who may assemble these layers before applying the device.

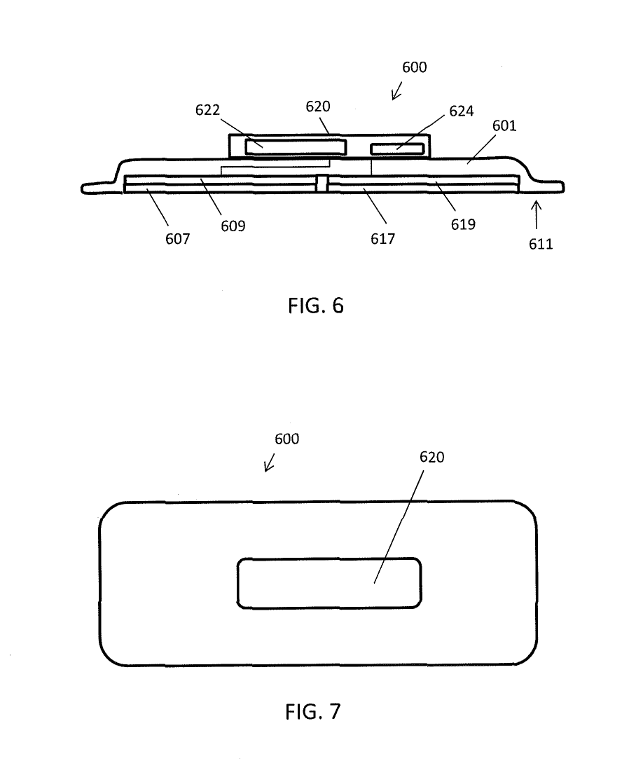

Another variation in which the one or more bioactive agents are applied by electrotransport is shown in FIGS. 6 and 7. In this example, the apparatus is also configured as a patch 600 including a first 607 and second 617 reservoir that is in contact with a controller 622 and power source 624, which may apply an electrical gradient to drive the bioactive agent(s) out of the apparatus and onto/into the skin. The patch may be adhesively 611 secured to the skin, and multiple bioactive agents (and particularly charged agents) may be incorporated into the two or more 609, 619 reservoirs. The electrical components may be housed in the applicator body 601 in a housing 620 region. The controller may be single-use or reusable, and may be programmed for continuous release or for periodic release (e.g., driving release with a predetermined, random, or on-demand profile, as described above). FIG. 7 shows a top view of this apparatus configuration; the patch may have any appropriate shape, as discussed above.

### Alternative Embodiments

Any of the apparatuses and methods described herein may combine thermal (heating/cooling) stimulation and chemical stimulation. In addition or alternatively an apparatus as described herein may instead or additionally apply mechanical (e.g., vibrational) energy to any, some, or all of the body regions described herein, including in particular the forehead. The mechanical energy may be low frequency (e.g., less than 1000 Hz, less than 900 Hz, less than 800 Hz, less than 700 Hz, less than 600 Hz, less than 500 Hz, less than 400 Hz, less than 300 Hz, less than 200 Hz, less than 100 Hz, less than 80 Hz, less than 50 Hz, less than 40 Hz, less than 30 Hz, less than 20 Hz, less than 10 Hz, less than 5 Hz, etc.) applied.

Any of the applicator apparatuses described herein having an applicator body may also be configured to connect (wired or wirelessly) to a controller, which may be handheld or wearable, e.g., running on or part of a smartphone, watch, tablet computer, or the like, or a dedicated apparatus. The controller may control the application of the modality (e.g., release of the chemical stimulation, application of the energy (thermal, mechanical, etc.), including the frequency applied, on/off, intensity, and any other stimulation parameters.

For example, disclosed system for enhancing sleep including an applicator that is adapted to connect to a forehead to apply stimulation to modulate sleep onset, sleep duration, sleep intensity, etc.

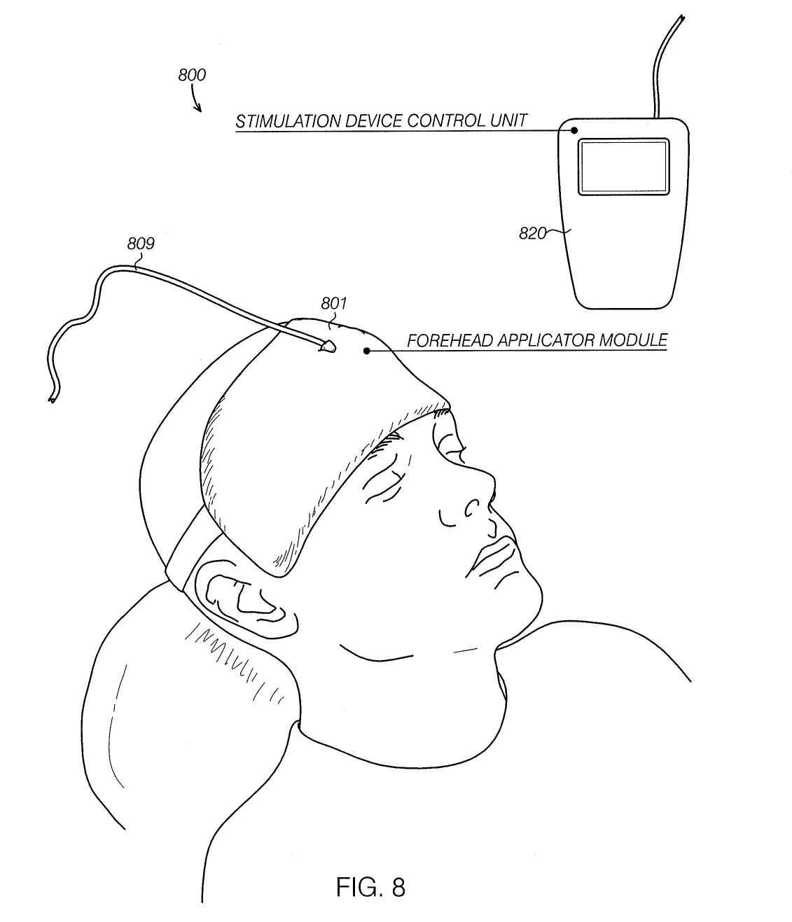

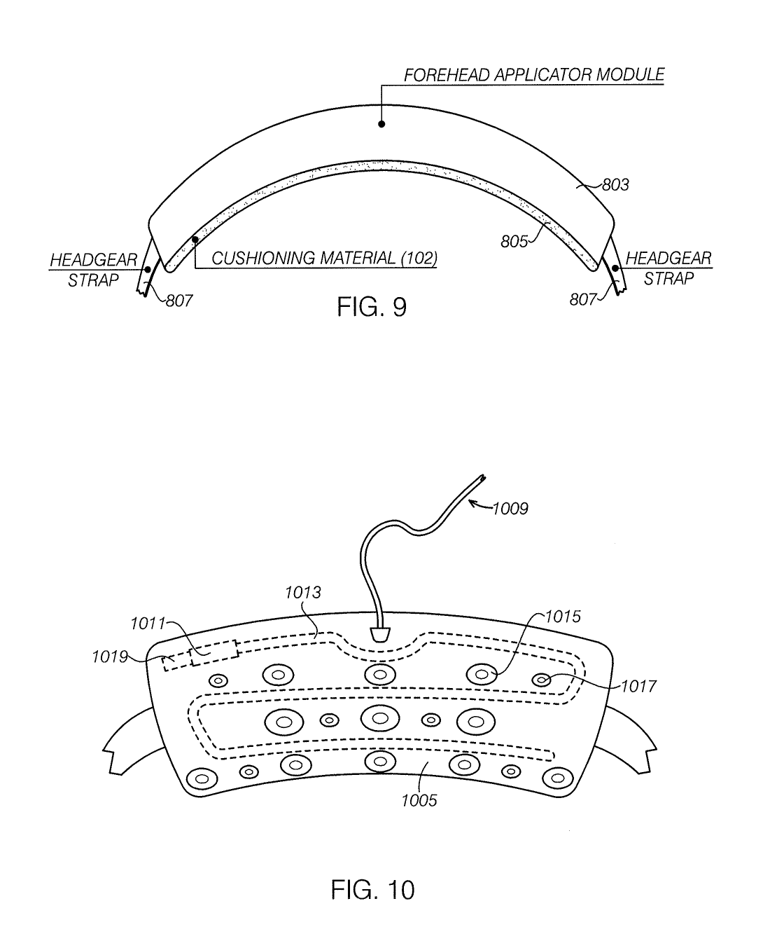

FIGS. 8 and 9 illustrate an example of an apparatus as described herein configured to apply frontal cortex stimulation 800. The device 800 includes a forehead applicator 801 for providing at least one form of stimulation (e.g., mechanical, chemical, electrical, etc.) to the wearer's forehead, a forehead applicator retainer 807 for maintaining the applicator 801 in a desirable position, and a stimulation device control unit (controller) 820 for controlling the applied modality. Also described below are various modalities, in addition or instead of chemical stimulation) that the applicator may provide to the wearer.

In this example, an applicator may be generally in a form that fits over the wearer's forehead. Applicator 801 may include components that provide one or more modality of stimulation to the forehead of the wearer. Applicator 801 includes a contact surface 105. The contact surface 105 is essentially the side of the forehead applicator module 101 that is in contact with the wearer's forehead. The contact surface 805 may fit snuggly against the wearer's forehead, and thus the contact surface may be cushioned to provide comfortable fit and wear. Good materials for the contact surface include but are not limited to pliable plastic, a gel-like material, or foam. In some instances the cushioning material not only covers the contact surface but may encase the entire forehead applicator module.

In some variations the applicator includes a plurality of discrete application elements in communication with the contact surface (behind and/or adjacent to). For example, in variations applying mechanical energy, one or a plurality of vibrational transducers may be in communication with the contact surface so that the vibrational energy can be transmitted through the contact surface to the wearer. A vibrational transducer may be a piezoelectric transducer, electromechanical transducer, or the like. In some variations the vibrational transducers are spread across the applicator; alternatively they may be clustered in one or more regions (e.g., above the eyebrow region, etc.)

In some variations, the applicator may include one or more internal channels. For example, the applicator may include a fluid channel. The movement of fluid through the channel(s) may provide a mechanical/vibrational sensation through the applicator. For example, the fluid may be applied in a pulsatile manner at any of the frequencies described above (e.g., less than 50 Hz, less than 10 Hz, etc., between 0.1 Hz and 50 Hz, between 0.1 Hz and 10 Hz, etc.). For example, as shown in FIG. 10, an applicator 801 may contain a contiguous channel 1013 throughout the plane of the applicator body. The channel may contain a liquid or a gel that can be circulated throughout the channel. In addition to or instead of cooling or heating the liquid or gel, the material may be circulated in a pulsed manner. The fluid or gel may be connected to an external pump, or an on-board pump 1011 may be connected to the channel. In this example, the pumping/circulating element 1011 may be connected to a controller 1019 that having circuitry and/or control logic or connecting to a separate controller (optional element 1009) for controlling the pumping/circulating element.

An applicator can alternatively or additionally include a variety of stimuli outputs 1015 (e.g., transducers), which may be flush, recessed or protruding and may be spread throughout the contact surface 1005 for delivering a burst of pressure (pulse) or a constant level of pressure. As mentioned, these transducers may be vibrating, or they may be inflatable elements, which may inflate and deflate. Inflating and deflating may be achieved with air or liquid within the contact surface body. When the applicator 801 is firmly fitted to the wearer's head, the transducer can provide vibration (e.g., a pulse of pressure) to all or certain areas of the forehead. An inflatable transducer can be completely inflated, completely deflated, or can provide be periodically inflated and deflated to provide a sensation at various regions on the wearer's forehead.

In another example, an applicator may contain components to deliver a vibration of a certain frequency, as discussed above. The vibration can be selected from a range of available frequencies and can be continuous or periodic. Further, vibrations can be provided to only certain regions on the wearer's forehead or the vibrations can be provided throughout the entire forehead applicator module. The user would be able to select the duration of the frequency or frequencies applied and able to select the length of the overall session.

In another example, an applicator may alternatively or additionally contain electrodes. Electrodes can deliver a range of electrical current at various regions of the contact surface of the forehead applicator module. A possible range of electrical current that can be delivered to the electrodes range from 0.5 mA to approximately 3 mA. The electrical current can be burst or a series of pulses for a set amount of time. In order for the electrical current to reach the wearer's forehead, some surface of the electrodes will likely be exposed from the contact surface of the forehead applicator module. Similarly here, a user will be able to select from an array of pre-programmed session that provide variable electrical outputs and for various amounts of time.

In another example, either static/constant field magnets or electromagnets can also be included in the applicator module and used to apply electromagnetic energy. In the case of electromagnetic fields, components such as coils, power supplies, pulse generators or frequency synthesizers, and filters may also be included. For example, an electromagnetic fields in the range of 5 Hz to 20 Hz may be applied. In this example, pre-programmed pulse sequences can be selected and implemented. A user may select the duration of the magnetic field as well as be able to select a range of possible frequencies to be applied.

In another example, an applicator may also have an auditory component. The auditory component can either play a noise cancelling sound, such as white noise, or be able to play soothing sounds, such as ocean waves, babbling brook, rainfall, and so forth. Also, a user can load sounds or music of his choosing to be placed in conjunction with other stimuli or by itself.

In yet another example, an applicator module can also include one or more sensors on or near the contact surface. The sensors can be used to detect and record physiological parameters of the wearer. Examples of useful physiological parameters include, but are not limited to body temperature, heart rate, and respiration.

An applicator may come in a number of standardized sizes. While a standard form of the forehead applicator module will come in a size that can fit the majority of wearers' foreheads, it conceivable that there are those that may require either a smaller or larger forehead applicator module in order to receive optimal stimulation of their frontal cortex. The forehead applicator module can also extend beyond the forehead region and cover past the wearer's eyebrow region or extend downward to cover the wearer's temples.

In any of the applicators descried, the applicator may be configured to fit snuggly against the wearer's forehead but should not be too restrictive. In some stances, elastic straps on either side of the forehead applicator, as shown in FIG. 1. It may also be possible for the wearer to adjust, either tighten and loosen, the straps to the desired comfort level.

An applicator may include a headgear-type retainer. The headgear maintains contact of the forehead applicator module to provide regional stimulation of the area in proximity of the frontal cortex. The headgear may be configured to allow the wearer to adjust the amount of contact pressure applied by the applicator and to adjust for comfortable wear, while still maintaining the position of the forehead applicator module over the wearer's forehead. A headgear may be made from a variety of materials. The material may be elastic or covered with an elastic material that would reduce increased contact pressure of the applicator to the wearer's forehead when stretched by the adjustable straps wrapping the circumference of the wearer's head. The adjustable straps can be constructed from any suitable material either exhibiting an elastic or static characteristic and incorporate any adjustable feature readily available such as Velcro, snaps, buttons, hooks, and so forth. In some variations, the adjustment feature may allow for macro-adjustments of the circumferential head size and secondary adjustment features to micro-adjust specific areas of the applicator to ensure optimal material in fixed sizes without adjustability, i.e. small, medium, or large.

A headgear may be reusable and separable from the applicator. Alternatively, the headgear may be integrated with the applicator. The headgear may also be single use. The headgear may be constructed of a singular piece to allow contact for regional stimulation. The headgear may be adjustable. In general, the headgear may be cushioned, particularly around the regions around the forehead applicator module. In other variations, the headgear may be constructed from multiple pieces for better contact and comfort of the wearer. The headgear may include a pocket or clips to secure the forehead applicator module against the wearer's head. In some examples, the headgear can be provided in different sizes that is able to accommodate various head circumferences.

As mentioned any of these apparatuses may include a controller, which may be separate from the applicator or integrated into the applicator. A separate controller may be handheld (or bedside) and may wireleses or by connection to a wire or wires communicate with a control receiver in the applicator. The control receiver may connect to the transducer(s) and may include a timer and control circuitry for executing the command controls from the controller. For example, a controller (control unit) may provide programs and controls to the applicator for providing a stimuli or a combination of stimuli. For example, a control unit may provide one or more of pre-programmed: temperature, pressure, vibrational, electrical, magnetic, and/or auditory sessions to be played alone or in combination. A user may be able to combine and mix various available stimuli to achieve optimal relaxation and aid in faster onset of sleep. A user may be able to upload preferred auditory programs to use in conjunction with the other provided stimuli.

As mentioned, any modality (e.g., stimulation or treatment modality) may be used alone or in combination, including temperature, tactile (e.g., mechanical stimulation such as vibration, pressure, etc.), electrical, magnetic, etc. For example, pressure may be applied to the wearer's forehead by inflating or deflating transducers to provide pressure to the wearer's forehead when the applicator is snuggly fitted to the wearer's forehead. Inflation and deflation (e.g., of localized regions on the applicator and/or the entire contact surface of the applicator) may be pulsatile and can be achieved with air or liquid and can be adjusted and controlled by the controller. Application of tactile (e.g., vibrational, pressure, etc.) can be located in on regions of the forehead applicator module that corresponds to acupressure points associated with relaxation. In some examples, a user can select pre-programmed pressure-delivering session or program his/her own pressure delivering session.

In some variations the applicator contains transducers able to generate a desired vibrational frequency alone or in combination with other modalities. For example, a frequency range of 4 Hz to 13 Hz can be used to stimulate the brain's production of Alpha, Mu, Theta, and Delta (electrical) waves. In some examples, a user can experience pre-program vibration session over a discrete amount of time. Further, a pre-programmed vibration session can be customized by the wearer.

An applicator may also be configured to apply electrical stimulation, alone or more preferably in combination with other modalities. Application of a small amount of electrical energy (e.g., current) can increase the production of Alpha waves in the brain. Acceptable currents range from 0.5 mA to approximately 3 mA. The stimulation device can include pre-programmed electrical stimulation over a discrete period of time. Further, electrical stimulation sessions can be coupled to sensor output to correspond to a wearer's sleep cycle. A user can also couple an electrical stimulation session with other available stimulation, such as an auditory output.

An applicator may also be configured to apply magnetic stimulation, alone or more preferably in combination with other modalities. An applicator may contain one or more magnets (static or electromagnets). Alternatively or additionally, the applicator may contain a series of components that can be used to induce an electromagnetic field. An exemplary frequency for a varying magnetic field applied may range from 5 Hz to 20 Hz, at a pulse train including pulse sequences of 20 to 30 pulses of 10 sec to 30 sec each, e.g., on a microsecond magnitude. Magnetic stimulation may include pre-programmed pulse sequences that can be played for a discrete number of pulse trains.

Any of the apparatuses described herein may also be configured to record biological parameters of the wearer. For example, the sensors located on the applicator may detect and transmit data on one or more physiological parameters (e.g., HR, skin temperature, EMG/EOG, skin surface potential, skin surface impedance, movement, etc.), and the control unit may receive, record and output these parameters via wired or wireless means.

For example, described herein are apparatuses for transdermally enhancing sleep in a subject, the device comprising: an applicator body; a skin-contacting surface on the applicator body, wherein the skin contacting surface includes one or more transducers configured to deliver a mechanical force (e.g., pressure, vibration) that is pulsatile and pulsed at a predetermined pulsing frequency (e.g., between 0.1 Hz and 1000 Hz, between 0.1 Hz and 200 Hz, between 0.1 Hz and 100 Hz, between 0.1 Hz and 50 Hz, between 0.1 Hz and 20 Hz, less than 100 Hz, less than 80 Hz, less than 50 Hz, less than 20 Hz, etc.). Any of these apparatuses may include a retainer (configured to hold the device against the skin). Any of these apparatuses may include a controller. In some variations the transducer is inflatable/deflatable. In some variations the transducer is a channel through which a fluid/gel is pulsed. In some variations, the transducer is a piezoelectric and/or mechano-electric transducer.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of enhancing sleep in a subject comprising:
positioning an applicator having a bioactive agent that chemically activates cold-sensitive, warm-sensitive, or warm-sensitive and cold-sensitive receptors on the subject's skin;
delivering the bioactive agent onto the subject's skin; and
chemically inducing a sensation of warming or cooling on the subject's skin to reduce sleep onset, improve sleep maintenance, increase sleep duration, reduce awakenings or increasing the ratio of deep sleep relative to light sleep in the subject.

2. (canceled)

3. (canceled)

4. The method of claim 1, wherein positioning comprises placing the applicator onto the subject's foot, forehead, or hand.

5. The method of claim 1, wherein positioning the applicator comprises placing an adhesive patch on the subject's skin, wherein the patch comprises the bioactive agent.

6. The method of claim 1, wherein delivering comprises passively delivering the bioactive agent onto the subject's skin.

7. The method of claim 1, wherein delivering comprises delivering the bioactive agent to the subject's skin by electrotransport.

8. The method of claim 1, wherein delivering comprises transdermally delivering the bioactive agent.

9. The method of claim 1, wherein delivering comprises delivering the bioactive agent and a permeation enhancer onto the subject's skin.

10. The method of claim 1, wherein delivering the bioactive agent onto the subject's skin comprises delivering comprises delivering at least one of: 2-arachidonoylglycerol, 5(S)-HETE, 12(s)-HpETE, 15(S)-HpETE, arachidonoylethanolamide (AEA or anandamide), capsaicin, a capsaicinoid, a cold receptor agonist, icilin (1-(2-hydroxyphenyl)-4-(3-nitrophenyl)-3,6-dihydropyrimidin-2-one), dihydrocapsaicin, eucalyptol, homoedihydrocapsaicin, leukotriene B4, menthol (d menthol, dl menthol, l menthol), a menthol analogue, nordihydrocapsaicin, N-arachidonoyldopamine (NADA), N-oleoyldopamine (OLDA), oleoylethanolamide (OEA), resiniferatoxin (RTX), a TRPA (transient receptor potential A) agonist, a TRPA1 agonist, a TRPA(transient receptor potential A) antagonist, a TRPA1 antagonist, a TRPM 2, 4, or 5 agonist, a TRPM 2, 4, or 5 (melastatin transient receptor potential 2, 4, or 5) antagonist, a TRPV1-4 (transient receptor potential vanilloid-1-4) agonist, a TRPV1-4 (transient receptor potential vanilloid-1-4) antagonist, a TRPM 2, 4, 5, or 8 (melastatin transient receptor potential 2, 4, 5, or 8) agonist, or menthol, a vanilloid, vanillyamide of n-nonanoic acid (NVA or PAVA), and a warm receptor agonist.

11. The method of claim 1, wherein delivering comprises sequentially delivering a plurality of bioactive agents.

12. The method of claim 1, wherein delivering comprises timed release of one or more bioactive agents to the subject's skin.

13. The method of claim 1, wherein delivering comprises delivering a plurality of bioactive agents to the subject's skin.

14. The method of claim 1, wherein inducing the sensation of warming or cooling on the subject's skin without substantially changing the skin temperature comprises changing the subject's temperature less than 0.2° F.

15. The method of claim 1, inducing a sensation of warming or cooling on the subject's skin without substantially changing the skin temperature comprises changing the subject's temperature less than 0.5° F.

16. The method of claim 1, wherein positioning comprises positioning on the subject when the subject does not have a fever.

17. The method of claim 1, wherein positioning comprises positioning on the subject when the subject's skin temperature is within 0.5° F. of 98.6° F.

18. The method of claim 1, wherein positioning comprises positioning the applicator on a subject suffering from a sleep disorder.

19. The method of claim 1, wherein positioning comprises positioning the applicator on a subject suffering from acute insomnia.

20. The method of claim 1, wherein positioning comprises positioning the applicator on a subject suffering from chronic insomnia.

21. The method of claim 1, wherein positioning comprises positioning the applicator on the subject's forehead without contacting the periorbital, or cheek regions of the subject's face.

22. A device for transdermally enhancing sleep in a subject, the device comprising:
an applicator body;
a skin-contacting surface on the applicator body, wherein the skin contacting surface is configured to transfer a bioactive agent to the skin of a subject without changing the skin temperature beneath the applicator surface by more than 0.5° F.; and
a bioactive agent on the skin-contacting surface, wherein the bioactive agent activates cold-sensitive, warm-sensitive, or warm-sensitive and cold-sensitive skin receptors,
wherein the bioactive agent is configured to be time-released by the skin-contacting surface.

23. (canceled)

24. The device of claim 22, wherein the applicator is configured as an adhesive patch.

25. The device of claim 22 or 23, wherein the applicator comprises a plurality of microdomains of different bioactive agents, and wherein groups of microdomains are configured to be released from the skin-contacting surface at different times.

26. The device of claim 22, further comprising a permeation enhancer on the skin-contacting surface.

27. The device of claim 22, further comprising a controller, a power source connected to the controller, and a reservoir of bioactive agent in communication with the skin contacting surface and in contact with the power source, wherein the controller is configured to apply power to deliver the bioactive agent by electrotransport.

28. The device of claim 22, wherein the bioactive agent comprises at least one of: 2-arachidonoylglycerol, 5(S)-HETE, 12(s)-HpETE, 15(S)-HpETE, arachidonoylethanolamide (AEA or anandamide), capsaicin, a capsaicinoid, a cold receptor agonist, icilin (1-(2-hydroxyphenyl)-4-(3-nitrophenyl)-3,6-dihydropyrimidin-2-one), dihydrocapsaicin, eucalyptol, homoedihydrocapsaicin, leukotriene B4, menthol (d menthol, dl menthol, l menthol), a menthol analogue, nordihydrocapsaicin, N-arachidonoyldopamine (NADA), N-oleoyldopamine (OLDA), oleoylethanolamide (OEA), resiniferatoxin (RTX), a TRPA (transient receptor potential A) agonist, a TRPA1 agonist, a TRPA(transient receptor potential A) antagonist, a TRPA1 antagonist, a TRPM 2, 4, or 5 agonist, a TRPM 2, 4, or 5 (melastatin transient receptor potential 2, 4, or 5) antagonist, a TRPV1-4 (transient receptor potential vanilloid-1-4) agonist, a TRPV1-4 (transient receptor potential vanilloid-1-4) antagonist, a TRPM 2, 4, 5, or 8 (melastatin transient receptor potential 2, 4, 5, or 8) agonist, or menthol, a vanilloid, vanillyamide of n-nonanoic acid (NVA or PAVA), and a warm receptor agonist.

29. The device of claim 22, wherein the skin-contacting surface is configured to cover a subject's forehead without contacting the periorbital, or cheek regions of the subject's face.
